(19) United States
(12) Patent Application Publication
(10) Pub. No.: US 2025/0377558 A1
(43) Pub. Date: Dec. 11, 2025

(54) OBJECTIVE REFRACTOR, COMBINED WITH MULTI-CHANNEL SUBJECTIVE REFRACTOR

(71) Applicant: RxSight, Inc., Aliso Viejo, CA (US)

(72) Inventor: John Kondis, Irvine, CA (US)

(73) Assignee: RxSight, Inc., Aliso Viejo, CA (US)

(21) Appl. No.: 19/297,028

(22) Filed: Aug. 12, 2025

Related U.S. Application Data
(63) Continuation-in-part of application No. 18/734,155, filed on Jun. 5, 2024.

(51) Int. Cl. G02C 7/02; G02B 27/00; H04N 23/55; H04N 23/56; H04N 23/68

(52) U.S. Cl. CPC G02C 7/027; G02B 27/005; G02B 27/0068; H04N 23/55; H04N 23/56; H04N 23/687; G02C 2202/22

(57) ABSTRACT

An autorefractor includes a point-like light source, to emit a light beam; a beam splitter, to direct the light beam; an aberration compensator optic, to receive the light beam from the beam splitter, to propagate the light beam with a compensating aberration to an eye of a patient, and to propagate a reflected light beam, reflected by the eye, to the beam splitter; wherein the beam splitter is configured to direct the reflected light beam, received from the aberration compensator optic; a camera, to receive the reflected light beam from the beam splitter, and to capture an image formed by the reflected light beam; and a controller, to determine a compensation indicator of the reflected light beam from the captured image, and to adjust the aberration compensator optic to improve the compensation indicator. In some embodiments, the above autorefractor can be combined with a multi-channel subjective refractor.

### TECHNICAL FIELD

This application is related to refractors, in more detail, to an objective refractor, optionally combined with a multi-channel, parallel-presenting subjective refractor.

### BACKGROUND

Refractors, or phoropters, have been employed by optometrists for a very long time for the purpose of determining the optimal vision correction for their patients. The historical refractor is an opto-mechanical system where the optometrist can change lenses of differing diopters in and out of the patient's viewing path of an eye chart sequentially and prompt the patient to report which lens provided the sharper image, the previous or the present one. Such refractors that rely on feedback from the patient can be classified as subjective refractors.

With recent technical developments, it became possible to perform objective measurements of the wavefronts of the patients' eyes by various techniques, using opto-electronic aberrometers from companies like NIDEK, WaveDyn, or many other providers. One motivation for introducing objective aberrometers was the belief that such measurements can be automated and therefore may eliminate the need for a trained optometric technician or the optometrist, thus saving precious time and cost. Another motivation was the belief that the high precision measurements of these aberrometers may determine the optimal refraction better and more reproducibly than “unreliable” subjective patient feedback. Much to the surprise of practitioners, however, objective aberrometers suffer from a frequent disagreement between the best refraction identified from the objective physical wavefront measurements and the subjectively best refractive condition reported by the patient. Therefore, there is an ongoing role for subjective refractors in an optometrist's practice. Remarkably, in spite of all the developments in opto-electronics, today's refractors/phoropters look startlingly close to the refractors used several decades ago, even a century ago. Puzzlingly, there has been minimal progress to apply the techniques of modem electro-optical devices to create new generations of phoropters, and to improve their performances.

### SUMMARY

In order to adapt some advantages and benefits of the modem opto-electronic devices for the present-day phoropter technologies, a multi-channel subjective refractor has been developed. Some embodiments of this multi-channel subjective refractor can comprise a first display to generate a first image; a second display to generate a second image; a first channel to refract the first image with a first channel refraction; a second channel to refract the second image with a second channel refraction; a beam combiner to receive and to combine the first image and the second image; and a shared channel, to receive the first image and the second image from the beam combiner to refract, in combination with the first channel, the first image with a first refraction; to refract, in combination with the second channel, the second image with a second refraction; and to present the first image with the first refraction and the second image with the second refraction to an eye simultaneously.

In some embodiments, a method of operating a multi-channel subjective refractor, the method comprises the steps of generating a first image with a first refraction and a second image with a second refraction with the multi-channel subjective refractor; presenting the first image with the first refraction and the second image with the second refraction simultaneously for an eye of a patient; and prompting the patient to identify the sharper of the first image and the second image.

In some embodiments, an objective refractor, or autorefractor can comprise a point-like light source, to emit a light beam; a beam splitter, to direct the light beam; an aberration compensator optic, to receive the light beam from the beam splitter, to propagate the light beam with a compensating aberration to an eye of a patient, and to propagate a reflected light beam, reflected by the eye, to the beam splitter; wherein the beam splitter is configured to direct the reflected light beam, received from the aberration compensator optic; a camera, to receive the reflected light beam from the beam splitter, and to capture an image formed by the reflected light beam; and a controller, to determine a compensation indicator of the reflected light beam from the captured image, and to adjust the aberration compensator optic to improve the compensation indicator.

In some embodiments, the above autorefractor can be combined with the above multi-channel subjective refractor.

### BRIEF DESCRIPTION OF THE DRAWINGS

FIGS. 1A-B show schematics of a multi-channel subjective refractor.

FIGS. 2A-B show embodiments of the multi-channel subjective refractor with no shared refraction in the shared channel.

FIGS. 3A-F show embodiments of the multi-channel subjective refractor with refraction in the first, second and shared channels.

FIGS. 4A-B show two images with different refractions.

FIG. 5 shows a binocular multi-channel subjective refractor.

FIG. 6 shows a head mounted binocular multi-channel subjective refractor.

FIG. 7 shows an automated multi-channel subjective refractor.

FIG. 8 shows steps of a method of using a multi-channel subjective refractor.

FIG. 9 shows sub-steps of the generating step.

FIG. 10 shows an implementation of the autorefractor.

FIG. 11 shows an implementation of the autorefractor.

FIG. 12 shows a sequence of image spots in the camera as the movable lens is moved.

FIGS. 13A-B show a sequence of images spots in the camera as the movable lens is moved (FIG. 13A), and as the rotatable Stokes lens is rotated (FIG. 13B).

FIGS. 14A-C show various adjustment sequences.

FIGS. 15A-C show an operation of the embodiment with a variable power lens.

FIGS. 16A-E illustrate another method, used by the autorefractor to determine the patient prescription.

FIGS. 17A-D show various ways to combine the autorefractor and the multi-channel subjective refractor.

FIG. 18 shows a particular embodiment of the combination of the autorefractor and the multi-channel subjective refractor in more detail.

### DETAILED DESCRIPTION

In traditional refractors, or phoropters, the patient is shown an eye chart with a first and a second refraction sequentially, and is prompted to choose/report the refraction that provided a sharper image: “Which was sharper: one or two” ?However, to answer this prompt, the patient must remember the visual quality of the previously shown refraction which is now not visible. Therefore, these sequential refractor designs and processes put a substantial mental and cognitive demand on the patient: an unwanted feature of any medical process. Second, for a large fraction of patients, especially older patients, the sequential presentation often leads to the patient asking the optometrist several times to flip back and forth between the refractions. This back-and-forth can be quite frustrating for all involved. Third, sequential processes are time consuming and use an excessive amount of the very precious chair time of the optometrist, thus being quite costly in time and money. Fourth and finally, in spite of all of this effort and cost, since the patient does not see the two, compared images simultaneously, the selection process still may not converge and select the optimal refraction.

A notable differentiator of a here-disclosed multi-channel subjective refractor 100 is that it presents the images with the to-be compared refractions simultaneously, side-by-side, or top-bottom. Such a simultaneous, or multi-channel presentation of refractions instantaneously eliminates the demand that the patient remembers the appearance of the previous image, and therefore removes the accompanying mental load, to the great relief of patient and optometrist alike. Second, comparing two images with both of them visible at the same time naturally eliminates the frustrating back-and-forth (“Which is sharper: one or two?”) with the optometrist. Third, such a simultaneous presentation saves very valuable chair time and allows the optometrist to see many more patients a day, and thus reduces cost and increases revenue. Fourth and finally, the process leads to the determination of the optimal refraction with a higher probability.

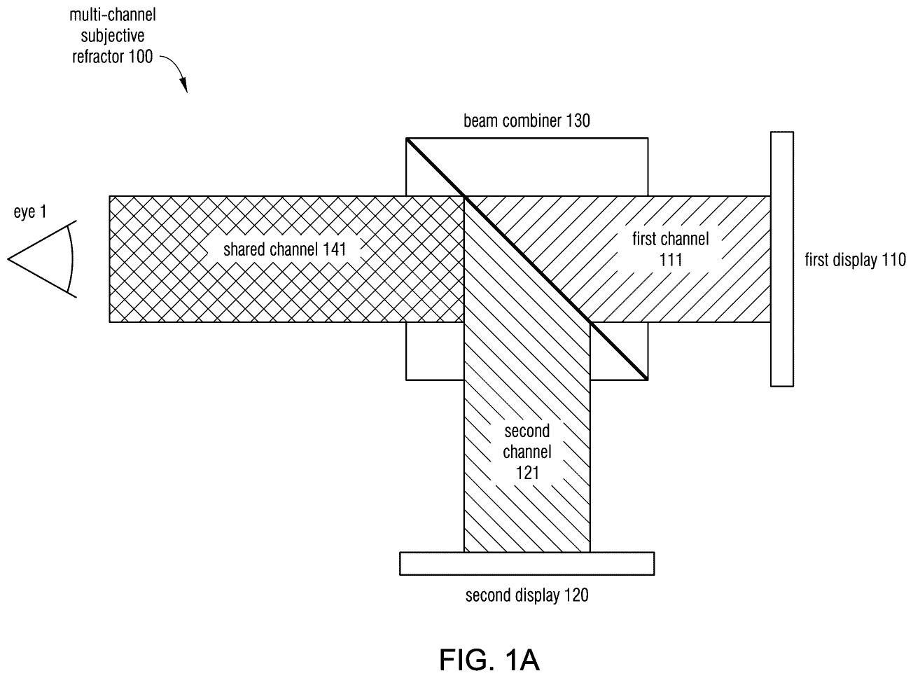

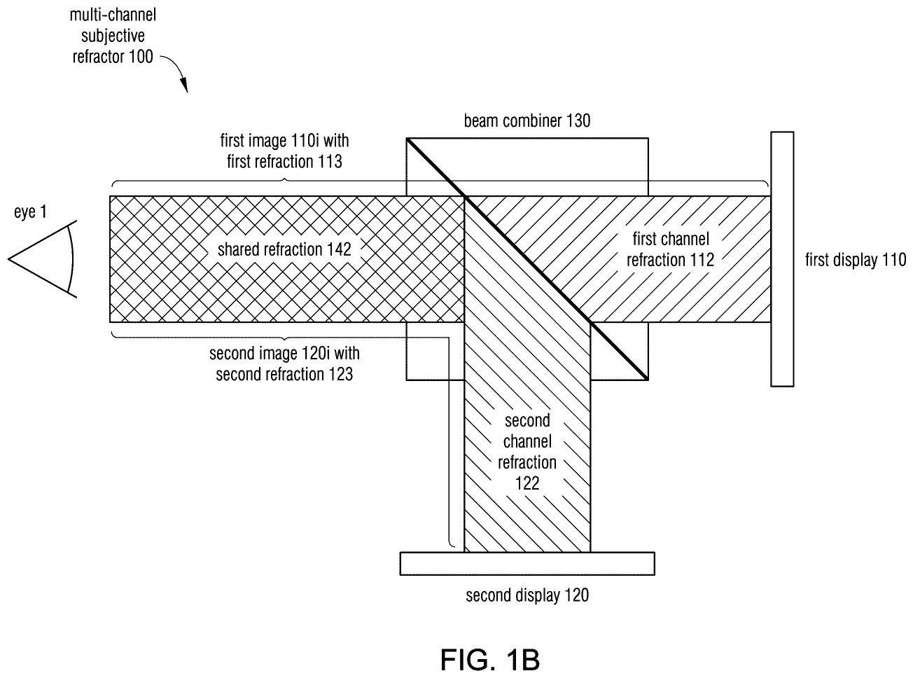

FIGS. 1A-B show that the above-described needs can be addressed by a multi-channel subjective refractor 100 that includes a first display 110 to generate a first image 110i; a second display 120 to generate a second image 120i; a first channel 111 to refract the first image 110i with a first channel refraction 112; a second channel 121 to refract the second image 120i with a second channel refraction 122; a beam combiner 130 to receive and to combine the first image 110i and the second image 120i; and a shared channel 141, to receive the first image 110i and the second image 120i from the beam combiner 130; to refract, in combination with the first channel 111, the first image 110i with a first refraction 113; to refract, in combination with the second channel 121, the second image 120i with a second refraction 123; and to present the first image 110i with the first refraction 113 and the second image 120i with the second refraction 123 to an eye 1 simultaneously.

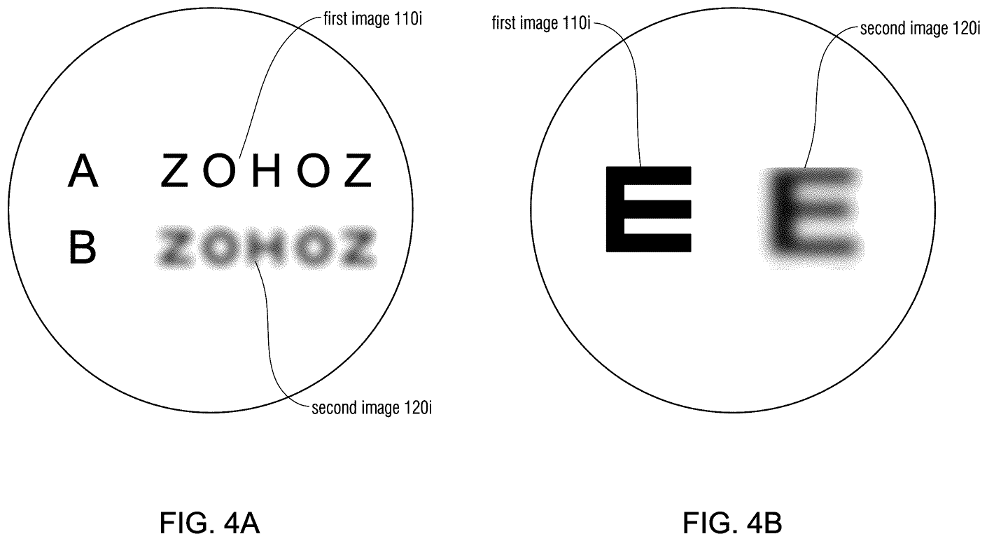

In operation, the patient is prompted to choose which of the simultaneously presented first image 110i with the first refraction 113 or the second image 120i with the second refraction 123 appears sharper. As discussed in relation to FIGS. 4A-B, following practical reasons and historical practices, often the first image 110i and the second image 120i are the same letters of an eye chart, and only their refractions 113 and 123 are different. This prompting for a choice between two, simultaneously presented images is repeated iteratively to scan the relevant refraction space. Which refraction is chosen as the sharpest by the patient can then be used by the optometrist to prescribe a spectacle lens.

Embodiments of the multi-channel subjective refractor 100 may have a patient-facing front that is similar to wavefront aberrometers. This patient-facing front can be sleeker than traditional refractors.

It is mentioned that the beam combiner 130 is often also called a beam splitter—the two names just reflecting forward and backward operation of the same prismatic structure, with its diagonal surface covered with partially reflecting coating. In some embodiments of the multi-channel subjective refractor 100, the first channel 111 and the second channel 121 can share an optical aperture, or axis, after being combined by the beam combiner 130. In some cases, they can also share an aperture.

Refraction is a broad category, relating to various wavefront modification of the generated first and second images 110i and 120i. In particular, in some embodiments of the multi-channel subjective refractor 100, the first channel refraction 112 comprises at least one of a first channel defocus and a first channel cylinder; the second channel refraction 112 comprises at least one of a second channel defocus and a second channel cylinder; the first refraction 113 comprises at least one of a first defocus and a first cylinder; and the second refraction 123 comprises at least one of a second defocus and a second cylinder. Broader definitions of refraction could also include higher order wavefront modifications. For completeness, while defocus is technically a measure of how far a detector plane is from the plane in which image points come into focus, it is closely related to optical power, sphere power, or simply sphere, all having diopters as their units. Defocus expressed as a power can be considered to be the amplitude of the negative of the defocus wavefront aberration which, if added to the system, would cause the image to land on the detector plane. The cylinder power, or astigmatism, can be considered the difference in defocus values required to get the system to focus along two orthogonal directions (axes) at the image plane. The cylinder also has an angular orientation, and thus is best represented by a vector that has both magnitude and direction. In general, the two main axes 1 and 2 are the main axes of curvature of the lens, and are 90° apart. The direction of the main axes, for practical purposes, can be the x and y axes, or two axes that are rotated by 45 degrees.

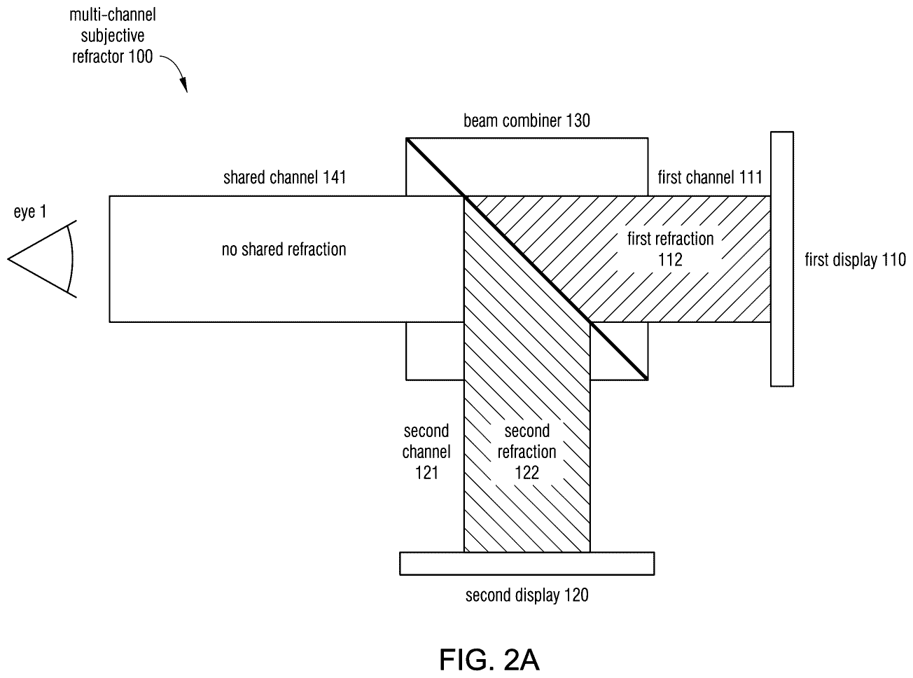

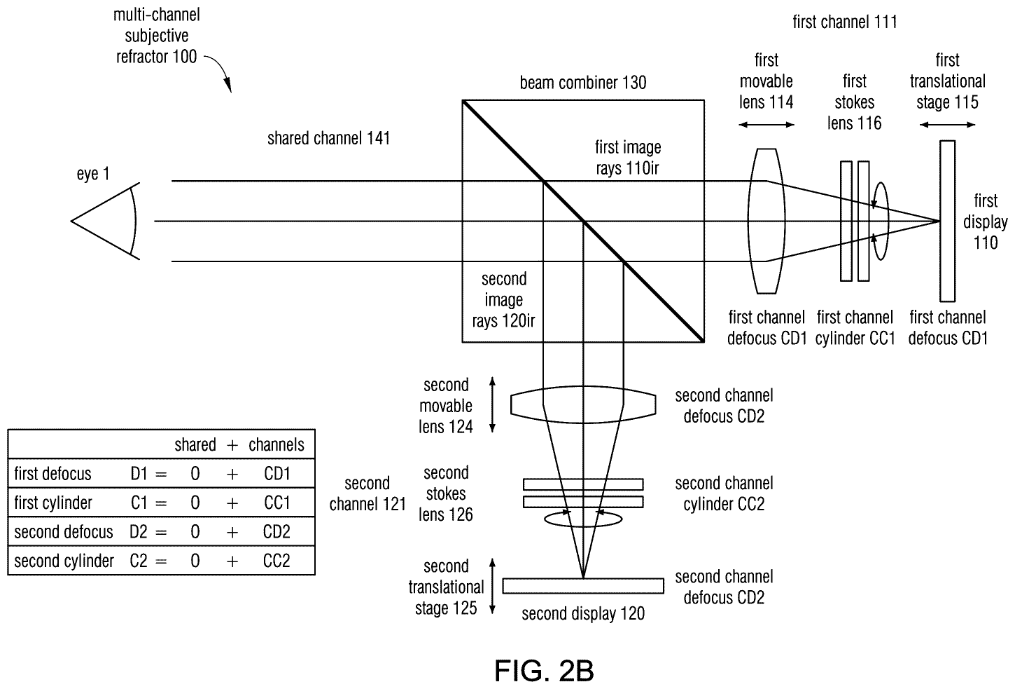

The scope of the above used “refractive combination” of the first and second channels 111/121 with the shared channel 141 is broad. In some embodiments, it may not even have any refractive contribution from the shared channel 141 itself. FIGS. 2A-B show such an embodiment of the multi-channel subjective refractor 100, where the shared channel 141 is not configured to refract the first image 110i and the second image 120i at all. In these embodiments, the first channel 111 provides the entire first refraction 113 for the first image 110i, and the second channel 121 provides the entire second refraction 123 for the second image 120i. In other words, in these embodiments the first channel refraction 112 equals the entire first refraction 113, the second channel refraction 122 equals the entire second refraction 123, and the refractive combination with the shared channel 141 receives no contribution from the shared channel 141.

FIG. 2B shows in a bit more detail that the first channel 111 can be configured to refract the first image 110i with the first defocus D1 and the first cylinder C1; and the second channel 121 can be configured to refract the second image 120i with the second defocus D2 and the second cylinder C2. As said above, in these embodiments, the first defocus D1 equals a first channel defocus CD1, the first cylinder C1 equals a first channel cylinder CC1; the second defocus D2 equals a second channel defocus CD2, and the second cylinder C2 equals a second channel cylinder CC2. In these embodiments, the first channel 111 may comprise at least one of a first movable lens 114, a first variable power lens, a first deformable mirror, a first phase modulator, and a first translational stage 115 to move the first display 110, to refract the first image 110i with the first defocus D1; and a first stokes lens 116, to refract the first image 110i with the first cylinder C1; and the second channel 120 may comprise at least one of a second movable lens 124, a second variable power lens, a second deformable mirror, a second phase modulator, and a second translational stage 125 to move the second display 120, to refract the second image 110i with the second defocus D2; and a second stokes lens 126, to refract the second image 120i with the second cylinder C2. Only some of the listed optical elements are shown in FIG. 2B.

The just-listed refractive combinations are summarized in the inserted table in FIG. 2B. Since the shared channel 141 is not configured to contribute any refraction, all four types of the refractions are generated solely by the first and second channel 111/121:

| | shared + channels |
|---|---|
| first defocus | D1 = 0 + CD1 |
| first cylinder | C1 = 0 + CC1 |
| second defocus | D2 = 0 + CD2 |
| second cylinder | C2 = 0 + CC2 |

For future reference, the shared defocus optionally caused by the shared channel 141 will be referenced as SD, and the optional shared cylinder as SC. From here on these abbreviations, or labels, will be used for such comprehensive refraction tables that characterize the various embodiments.

The “Stokes pair” or simply “Stokes lens” refers to a pair of cylindrical lenses of equal and opposite cylindrical power that can be rotated relative to each other about their shared optical axis. Their azimuthal positions can create a cylinder, or astigmatism, with a magnitude within a range, and with the cylinder direction pointing to any desired cylinder angle, by the suitable rotation of the two cylindrical lenses.

The above-described refractions can be described and determined by ray tracing, or following rays emitted by representative points of the first and second displays 110/120, such as the shown first image rays 110ir and second image rays 120ir. It is worth noting that in the shown embodiment, the first image rays 110ir and the second image rays 120ir are approaching the eye 1 in a nominally collimated manner, in order to make the multi-channel subjective refractor 100 project the displayed images as distant objects. In addition, optical rays propagating from the displays 110/120 are preferably telecentric, so that the apparent size of the projected images doesn't change as the images are defocused.

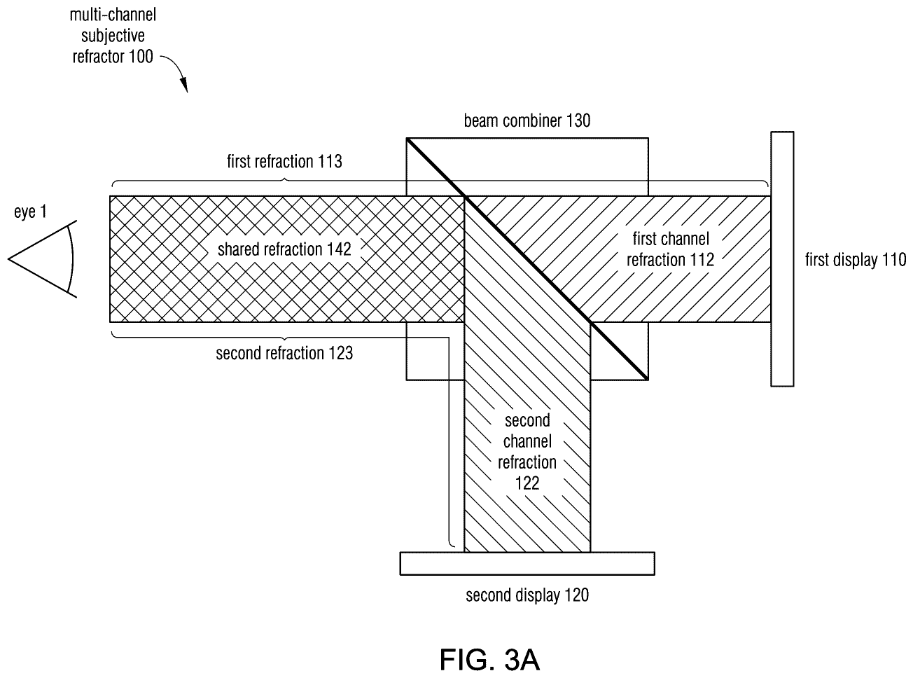

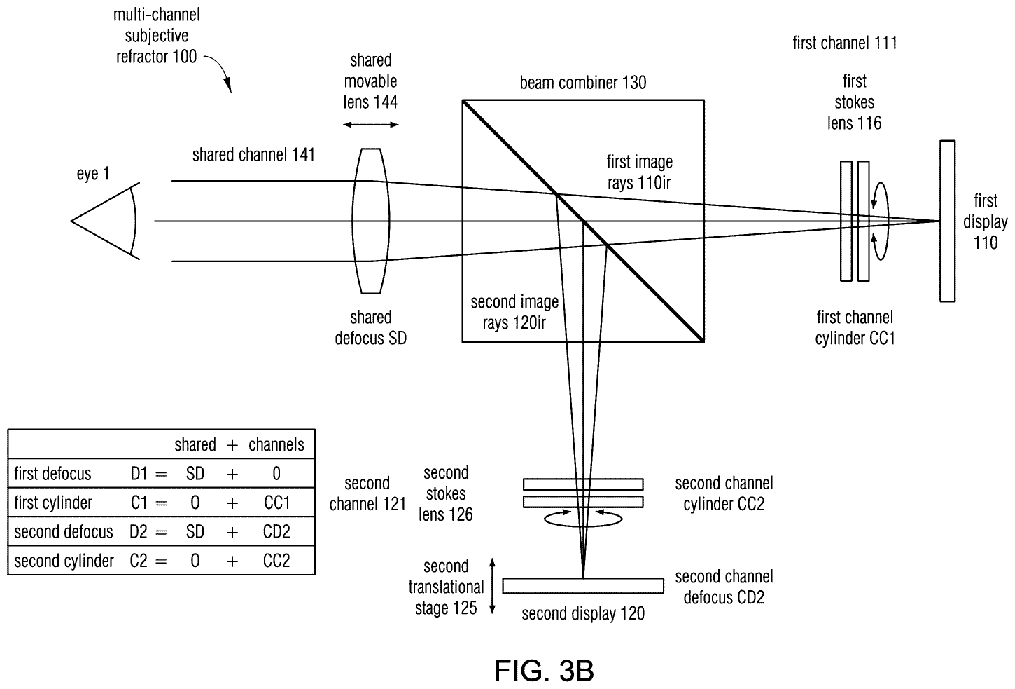

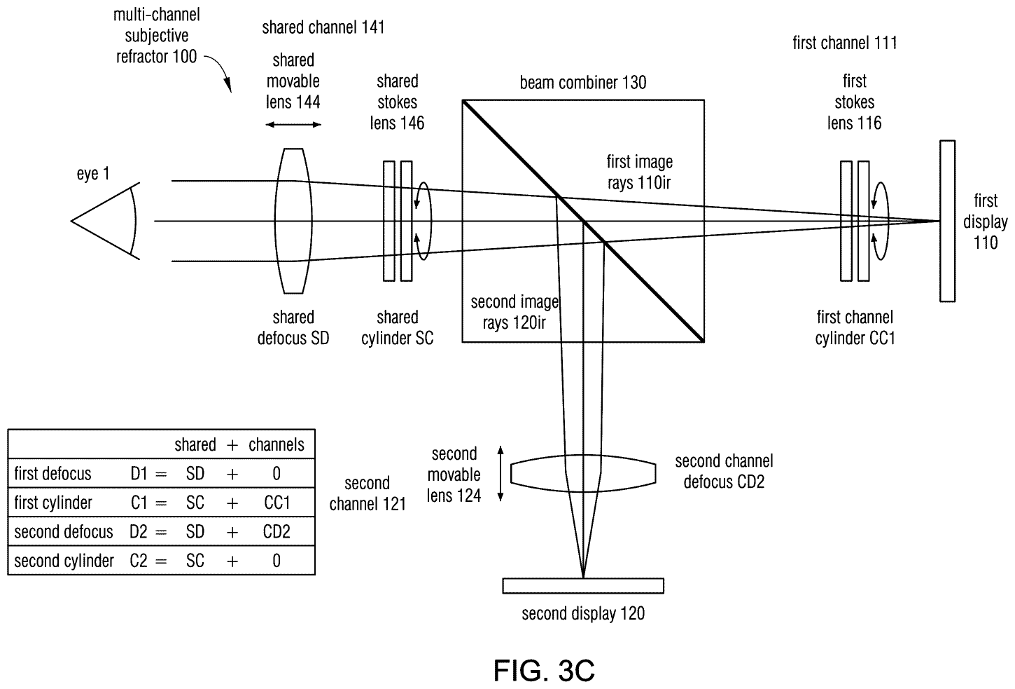

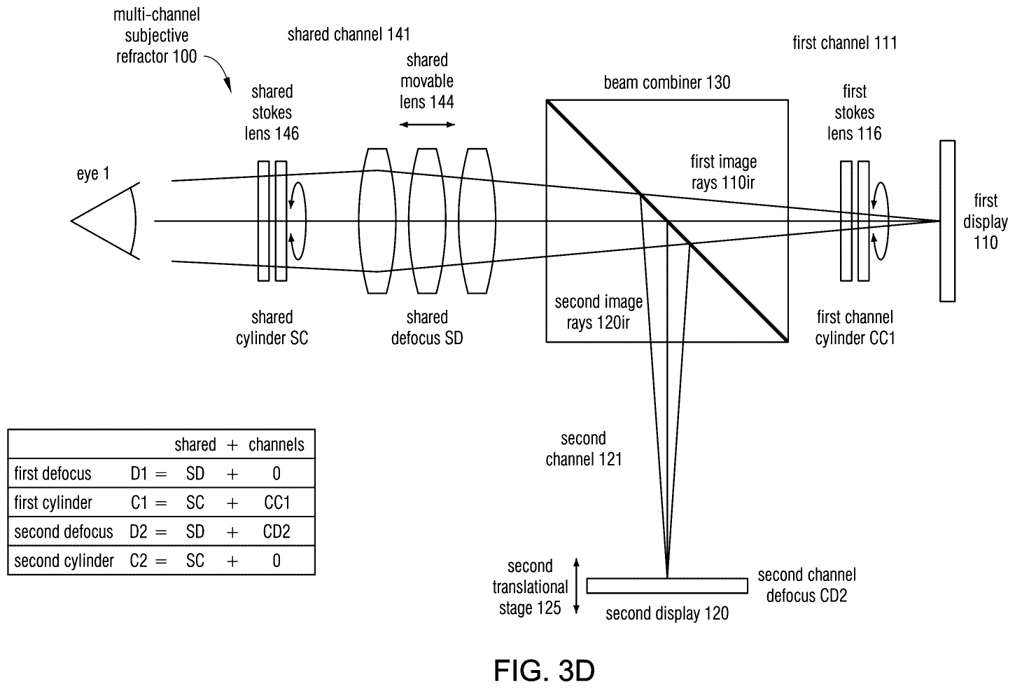

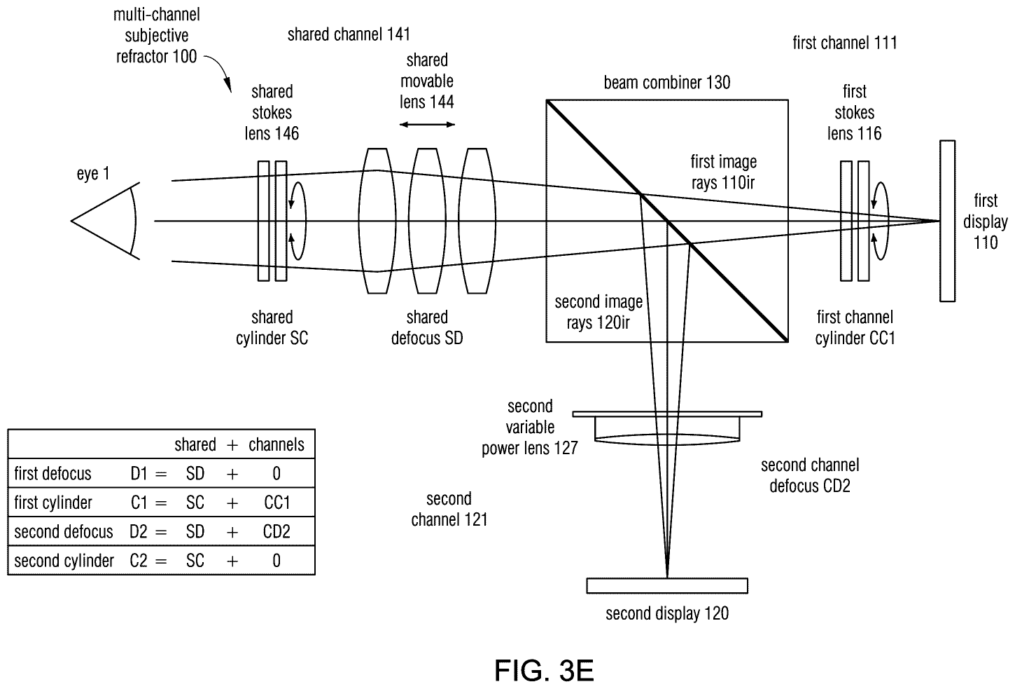

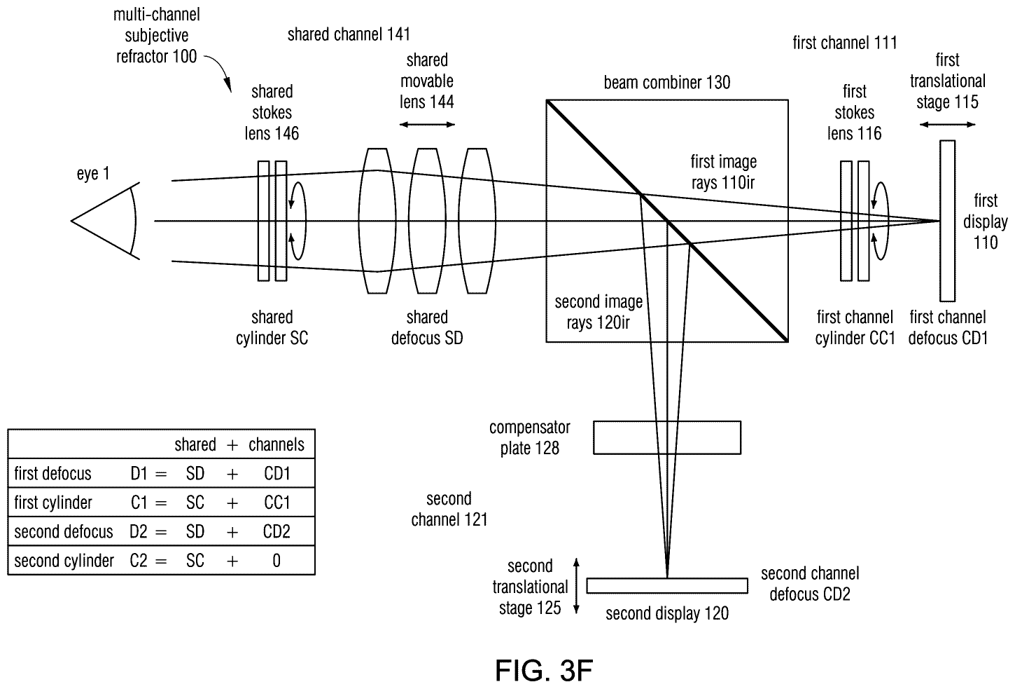

FIGS. 3A-F show several embodiments of the multi-channel subjective refractor 100 where the shared channel 141 is configured to refract the first image 110i and the second image 120i with a shared refraction 143 that includes at least one of a shared defocus SD and a shared cylinder SC. As shown in FIGS. 3B-F, in these embodiments, the shared channel 141 may include at least one of a shared movable lens 144, a shared variable power lens, a shared deformable mirror, or a shared phase modulator, to refract the first image 110i and the second image 120i with the shared defocus SD; and a shared Stokes lens, deformable mirror, or phase modulator 146, to refract the first image 110i and the second image 120i with the shared cylinder SC. In some of these multi-channel subjective refractors 100, the first channel 111 may include at least one of a first movable lens 114, a first variable power lens, a first deformable mirror, a first phase modulator, or a first translational stage 115 to move the first display 110, to refract the first image 110i with the first channel defocus CD1 that combines with the shared defocus SD to create the first defocus D1; and a first stokes lens 116, to refract the first image 110i with the first channel cylinder CC1 that combines with the shared cylinder SC to create the first cylinder C1; or the second channel 121 may include at least one of a second movable lens 124, a second variable power lens, a second deformable mirror, a second phase modulator, or a second translational stage 125 to move the second display 120, to refract the second image 120i with the second channel defocus CD2 that combines with the shared defocus SD to create the second defocus D2; and a second stokes lens 126, to refract the second image 120i with the second channel cylinder CC2 that combines with the shared cylinder SC to create the second cylinder C2. Accordingly, a generic summary of these refractive contributions can be summarized in this refraction table:

| | shared + channels |
|---|---|
| first defocus | D1 = SD + CD1 |
| first cylinder | C1 = SC + CC1 |
| second defocus | D2 = SD + CD2 |
| second cylinder | C2 = SC + CC2 |

In the embodiments of FIGS. 2A-B, the shared channel 141 did not contribute any refraction SD or SC to the “combined refractions”. Somewhat analogously, in the embodiments of FIGS. 3A-F, one of the first channel 111 or second channel 121 may not be configured to refract the corresponding first image 110i or second image 120i at all, and thus may not contribute to the combined refractions. In those cases, the shared refractions will produce the entirety of the first or second refractions, whichever has no channel refraction.

Here, a general note on the design. The human eye of patients can exhibit refractive deficiencies from −20 diopters to +20 diopters in defocus, in some extreme cases even more. Therefore, refractors also have to be able to cover such very wide ranges of diopters to provide comprehensive utility for the optometrists. However, covering such a wide power range requires a movable lens with a long moving stage, or a wide collection of lenses, to be inserted into the optical path with corresponding mechanics, or a variable power lens with a wide range of power variation, or a movable stage with long rails to move the display. Each of these engineering solutions takes up a lot of space, requires a number of moving parts, and increases the cost. In the “no shared refraction 143” multi-channel subjective refractors 100 of FIGS. 2A-B, both the first channel 111 and the second channel 121 have to be able to cover such a wide range of refractions with corresponding expensive and expansive optical designs. This makes the size of these refractors 100 big and the price high. In contrast, in the multi-channel subjective refractors 100 of FIGS. 3A-F which have a shared refraction 143, only the shared channel 141 needs to cover the wide range of refractions, and the first channel 111 and second channel 121 only need to cover a narrow differential refraction which is the narrow refraction difference between the first refraction 113 and the second refraction 123, presented to the patient as the comparison steps or intervals. This refraction can vary in a much narrower range, such as a defocus range of −1D to +1D. Therefore, in such shared refraction embodiments of the multi-channel subjective refractor 100, only the optical elements in the shared channel 141 cover the wide range of sphere and cylinder powers, and are therefore expansive and expensive, while the small and narrow-range optical elements in the first channel 111 and second channel 121 cover only the much-narrower differential refraction range, and are therefore small and much less expensive. Such shared refraction embodiments therefore offer benefits for an optometry practice.

In an example, the shared channel 141 can be configured to cover a wide defocus range of −20D to +20D, while both the first channel 111 and the second channel 121 can be configured to cover only the narrow differential defocus range of −1D to +1D. In another example, the shared channel 141 can be configured to cover the wide defocus range of −20D to +20D, the wide cylinder power range of −10, or −15, cylinder D to +10, or +15, cylinder D, and the wide cylinder angle range from 0 to 180 degrees, whereas the first channel 111 and the second channel 121 can be configured to cover only the differential defocus range of −1.5D to +1.5D and the differential cylinder power range of −1 cylinder D to +1 cylinder D. Similarly, in some embodiments of the multi-channel subjective refractor 100 at least one of the first channel defocus CD1, the first channel cylinder CC1, the second channel defocus CD2 and the second channel cylinder CC2 are all less than two diopters, where “diopter” refers inclusively to both sphere and cylinder diopters.

Of course, in some embodiments of the multi-channel subjective refractor 100, at least one of the first channel 111 and the second channel 121 can cover wide diopter ranges. For example, at least one of the first channel defocus CD1 and the second channel defocus CD2 can cover a range of +/−15 D, in some cases +/−20 D and at least one of the first channel cylinder CC1 and the second channel cylinder CC2 can cover +/−10 D, in some cases +/−15 D.

A further simplification can be achieved in some embodiments of the multi-channel subjective refractor 100 by using a design where one of the first channel 111 or the second channel 121 is not configured to refract the corresponding first image 110i or second image 120i at all. In such embodiments, one of the first refraction 113 or the second refraction 123 simply equals the shared refraction 143, and the differential refraction is generated only by the other, refracting channel. Finally, in some embodiments, different portions of the differential refraction can be implemented in different channels. For example, the differential defocus can be implemented only in the first channel 111 as CD1, but not accompanied by a differential channel cylinder (i.e. CC1=0), while the differential cylinder is implemented in the second channel 121 as CC2, wherein the second channel 121 does not have any defocus: CD2=0.

FIGS. 3B-F show several embodiments of such “shared-refraction plus distributed differential refraction” designs. In particular, FIG. 3B shows an embodiment where the first channel 111 is only refracts the first image 110i with a first channel cylinder CC1 via a first stokes lens 116, while the second channel 121 is configured to refract the second image 120i with a second channel defocus CD2 via a second translational stage 125 that moves the second display 120 along the axis. In some cases, the second channel 121 can further include a second stokes lens 126 to also refract the second image with a second channel cylinder CC2, as shown. In this embodiment, the shared channel 141 includes a shared movable lens 144 to refract both images with a shared defocus SD, but not with a shared cylinder SC. The summary refraction table reads as:

| | shared + channels |
|---|---|
| first defocus | D1 = SD + 0 |
| first cylinder | C1 = 0 + CC1 |
| second defocus | D2 = SD + CD2 |
| second cylinder | C2 = 0 + CC2 |

FIG. 3C shows another exemplary embodiment of the multi-channel subjective refractor 100, where the shared channel 141 includes both the shared movable lens 144 to refract the first and second images 110i/120i with the shared defocus SD, and a shared stokes lens 146 to refract the first and second images 110i/120i with a shared cylinder SC. In addition, the first channel 111 has the first stokes lens 116 to refract the first image 110i with the first channel cylinder CC1, and the second channel 121 has a second movable lens 124 to refract the second image 120i with the second channel defocus CD2. The corresponding refraction table reads as:

| | shared + channels |
|---|---|
| first defocus | D1 = SD + 0 |
| first cylinder | C1 = SC + CC1 |
| second defocus | D2 = SD + CD2 |
| second cylinder | C2 = SC + 0 |

FIG. 3D shows yet another implementation of the multi-channel subjective refractor 100. Here the shared channel 141 again includes the shared movable lens 144 to refract the first and second images 110i/120i with the shared defocus SD, and also includes the shared stokes lens 146 to refract the first and second images 110i/120i with a shared cylinder SC. The first channel 111 has the first stokes lens 116 to refract the first image 110i with the first channel cylinder CC1, and the second channel has the second translational stage 125 to translate the second display 120 to refract the second image 120i with the second channel defocus CD2. FIG. 3D shows that in this description the term “lens” is used in a broad, inclusive way to refer to any lens assembly that satisfies the overall function of a lens. Lens assemblies are often used in place of individual lenses to optimize the system performance in face of other challenges. These include, for example, the need to reduce chromatic aberration by using a crown-flint doublet in place of a single lens as the shared movable lens 144, as shown in FIG. 3D. These lens assemblies are also referred to as achromats, or as cemented doublets. Other lens assemblies may be used to increase contrast sensitivity or to reduce thermal expansion coefficients. The refraction table once again reads:

| | shared + channels |
|---|---|
| first defocus | D1 = SD + 0 |
| first cylinder | C1 = SC + CC1 |
| second defocus | D2 = SD + CD2 |
| second cylinder | C2 = SC + 0 |

FIG. 3E shows yet another embodiment of the multi-channel subjective refractor 100. The main difference relative to FIG. 3D is that in the second channel 121, the second channel defocus CD2 is generated by a second variable power lens 127, instead of the second movable lens 124, or the second translational stage 125 for the second display 120. There is a wide and growing selection of innovative variable power lenses 127. These include fluid filled lenses, where the curvature and therefore the power of the lens is varied by controlling the amount and possibly the spatial distribution of the fluid by applying pressure, or pumping fluids from fluid reservoirs. Variable power lenses 127 can also be formed with suitably engineered and spatially distributed liquid crystals where the focus is varied with applying an external voltage. Yet other solutions may also be used, like Alvarez lenses, where two optical elements with a cubic or more complex profile are rotated relative to each other. Some embodiments may achieve the effect of variable power by using a deformable mirror. The refraction table of FIG. 3E is the same as for FIG. 3D, as shown.

Finally, FIG. 3F shows yet another embodiment of the multi-channel subjective refractor 100. This again has the shared movable lens (assembly) 144 to refract the first and second images 110i/120i with a shared defocus SD, and the shared stokes lens 146 to refract the images with a shared cylinder SC. The first channel 111 includes a first translational stage 115 to translate the first display 110 to refract the first image 110i with the first channel defocus CD1, and the first stokes lens 116 to refract the first image 110i with the first channel cylinder CC1. The second channel 121 includes the second translational stage 125 to translate the second display 120 to refract the second image 120i with the second channel defocus CD2. In addition, this embodiment may include a compensator plate 128 that can be included to compensate higher order aberrations or chromatic aberrations that emerge between the first image 110i and the second image 120i because they propagate through different optical elements. This embodiment includes translational stage 115 and 125 in both channels, and thus contains more degrees of freedom than minimally required. Such redundant elements in this and in earlier-described embodiments may be included to provide added control and to further minimize unwanted optical effects, such as chromatic aberrations or higher order aberrations. The refraction table for the embodiment of FIG. 3F reads as:

| | shared + channels |
|---|---|
| first defocus | D1 = SD + CD1 |
| first cylinder | C1 = SC + CC1 |
| second defocus | D2 = SD + CD2 |
| second cylinder | C2 = SC + 0 |

The above-described large number of embodiments of the multi-channel subjective refractor 100 are examples of configuring the first channel 111, the second channel 121 and the shared channel 141 to enable an independent adjustment of the first refraction 113 and the second refraction 123. Before moving on, it is also mentioned that embodiments of the multi-channel subjective refractor 100 can be collimating, as the multi-channel subjective refractor 100 can image the displays 110/120 to infinity. Typically, this effect is achieved by emitting a collimated beam through an exit pupil towards the eye 1 of the patient, as shown with the tracing of the first and second image rays 110ir and 120ir. Further, in some simple optical designs, the projected size of the objects may change as the focus is changed. However, this can lead to an error in the patient's perception as a larger image is often perceived as “clearer” by a patient. Some embodiments of the multi-channel subjective refractor 100 preempt this problem by making use of an optics (first channel 111, second channel 121 and shared channel 141) that is telecentric, and thus the apparent size of the projected object (images 110i/120i) does not change with a changing of the focus. Other embodiments do not need such a telecentric optic, as the size of the images 110i/120i can be also changed electronically on the displays 110/120.

It is mentioned that recently a new class of intraocular lenses has been introduced that are light adjustable non-invasively after implantation. For these light adjustable lenses (LALs), the optometrist performs a refraction measurement after the implantation to measure the difference of the refraction of the implanted LAL from the planned, or patient-preferred refraction. These differences are typically small, typically in the 0.5 D-1.5 D range. Therefore, some embodiments of the multi-channel subjective refractor 100 that are intended to be used only in conjunction of the LAL adjustment procedure, can be made much smaller, since the movable lenses 114/124/144 and the translational stages 115/125 need to cover only the limited range of optical refractive powers of about −2 D to +2 D, instead of the general purpose refractors that typically cover much wider ranges up to −20 D to +20 D. Smaller multi-channel subjective refractors 100 with limited measurement and adjustment ranges naturally cost less, and thus can bring the medical benefits of the LAL to a wider range of patients.

For completeness it is mentioned that in embodiments where the covered diopter range is wider, additional optical design elements may need to be introduced. For example, if the first channel 111, the second channel 121, or the shared channel 141 is designed to cover a wide diopter range of −10D/+10D, then the beam may need to be deflected out by a mirror toward a Porro prism, from where the returning beam is reflected back into the channel by a second mirror. In some designs, the Porro prism can provide coverage for the −10D/+10D diopter range by being moved by 3-5 inches on a moving stage.

FIGS. 4A-B show that embodiments of the multi-channel subjective refractor 100 can be configured to present the first image 110i with the first refraction 113 and the second image 120i with the second refraction 123 simultaneously as an up-down image pair (FIG. 4A), or a side-by-side image pair (FIG. 4B), or any other positioning of an image pair. Corresponding to these presentation types, multi-channel subjective refractors 100 can also be called dual field refractors, simultaneous refractors, parallel refractors, or side-by-side refractors. The presented images can be of a wide variety, including actual photos and pictures, or a line of a Snellen chart, or individual letters, for example the often used capital E, or any kind of eye chart, or a log MAR chart, or an armed force target. The patient's communication may be made clearer by attaching a label to the first image 110i and the second image 120i, e.g. the letters A and B in FIG. 4A.

While the electronics of the multi-channel display 100 clearly enables the first display 110 and the second display 120 to generate different first and second images 110i and 120i, the patient may find comparing identical images with different refractions easier. Also, showing the same line of an eyechart only with different refractions for comparison is the practice optometrists have been following historically. Therefore, in many embodiments of the multi-channel subjective refractor 100 the first image 110i and the second image 120i may be the same, and can include one or more letters of an eye chart, of a Snellen chart, of an army target, or any other figure of an examination chart, which is not necessarily alphanumerical.

FIGS. 4A-B show clearly a key aspect of the multi-channel subjective refractor 100: that the patient is presented simultaneously, in parallel, with the images to be compared. The images are separated not in time, but only in space. As discussed, such refractors 100 (1) eliminate the mental, cognitive pressure on the patient to keep the memory of the visual clarity of previous images active; (2) enable a more accurate comparison because the two images are presented simultaneously and can be directly compared; (3) eliminate the frustrating repeated “back and forth” requests by patients to the optometrist/OD; and (4) greatly reduce the chair time, or time in the lane, because the comparison proceeds much faster, thereby substantially improving the workflow and economy of the optometrist office.

The process of exploring the relevant refraction space can be further accelerated by designing multi-channel subjective refractors 100 that include one or more additional channels, configured to present one or more additional images (beyond the first and second images 110i/120i) with corresponding refractions simultaneously for the eye. Such multi-channel subjective refractors 100 can thus present three, four, or more images simultaneously for the patient, who is then prompted to choose the sharpest image from the three, four or more images.

Here we make some comparative remarks. As known, the human eye refracts light with different wavelengths with slightly different indices of refraction. This is called dispersion, or chromatic aberration. The changing refractive index causes the optical power to change with wavelength. The difference of the eye's optical power between red and green light is approximately 0.5 D in the human eye. As a consequence, if the red component of an incoming light is focused on the retina, then the green component is focused about 0.2 mm in front of the retina. This fact is used in the so-called duochrome eye tests, when half of the black letters on the optical chart are presented over a red background and half of them over a green background. The effect of the eye's chromatic aberration or dispersion is that if the letters over the red background appear sharp, then the letters over the green background will appear blurry; and vice versa. The underlying theory is that the red and green wavelengths project objects that have an effective difference in refraction of about 0.5 D between them. The red and green colors are approximately symmetric on either side of the yellow color, thought to be key to optimize vision. Therefore, the patient is presented by a sequence of lenses and asked whether the letters over the red or green background appear sharper. The optometrist keeps changing the lenses until equal sharpness is achieved for the letters over the red and green backgrounds, which is taken as a sign that the yellow light is best focused on the retina.

This duochrome test is qualitatively different from the here-described method in several aspects. (1) The duochrome device presents two letters with equal optical power, or refraction, for the eye whereas embodiments of the multi-channel subjective refractor 100 present two letters with different refractions.
(2) In the duochrome test, the apparent difference of refractions is not generated by the device, but by the eye's chromatic aberration only, whereas embodiments of the multi-channel subjective refractor 100 do not rely on the eye's chromatic aberration as they typically present the letters over a white background.
(3) The duochrome test is useless for pseudophakic eyes with implanted diffractive IOLs, because those IOLs has minimal or negligible chromatic aberration. Some non-diffractive IOLs can also have lower chromatic aberration. In contrast, the multi-channel subjective refractor 100 can be very helpfully utilized to refract eyes with IOLs, especially light adjustable IOLs (LALs), where the refraction is an important step of the post-operative lens adjustment.
(4) The goal of the duochrome test is to achieve the patient reporting equally clear vision of the presented two letters, whereas a goal of the multi-channel subjective refractor 100 is the patient reporting one letter being clearer than the other one. Often, it is easier to notice and report a visual difference than to conclude that the visions of two eyes are equally clear.
(5) The duochrome test is not capable of testing and measuring cylinder.
(6) The duochrome method cannot easily change the difference between the two presented refractions unless the wavelengths are changed. The chromatic aberration between 550 nm (green) and 650 nm (red) is approximately 0.5 D. If one wanted to change the power difference, the wavelengths of the projections would have to be changed (for example by changing pigments in the ink or changing the emissive source of an electronic display), which is generally not easy to do with standard materials.
(7) Patients may be confused when presented with objects of two different colors and asked to specify the clearer one. One color may be more pleasant than the other or may appear “brighter” to the patient, influencing the patient's choice. Also, eyes are naturally more sensitive to green wavelengths than red, leading to another potential source of error.
(8) Neural detection and processing differences between green and red signals within the retina and optic nerve may also lead to a source of error in the measurement.
(9) Red-and-green duochrome methods won't work well if the starting state of the refractor is not already close to the patient's prescription. Far away from the ideal refraction, picking the sharper image may be difficult and error-prone for the patient. For this reason, duochrome methods are primarily used only for a potential “final adjustment” of refraction after the refraction has already largely been determined.

Some of these differences are well demonstrated in embodiments of the multi-channel subjective refractor 100, where the first display 110 generates the first image 110i with approximately or precisely the same background color as the second display 120 generates the second image 120i, while the first refraction 113 is different from the second refraction 123.

Yet another distinctive aspect of the multi-channel subjective refractor 100 is that it enables evaluating and optimizing vision experiences for patients at patient-preferred distances as part of prescribing glasses, or determining LAL adjustments. Some patients prefer optimized near vision, at distances of about 40 cm, others optimized distance vision at 6 meters, but in office settings it is more and more customary to optimize vision at intermediate distances, such as at 60 cm-70 cm. In a traditional optometrist lane, the measurement distances are fixed, whereas with the multi-channel subjective refractor 100 the optometrist can simulate the visual experience at any distance according to the patient's preference.

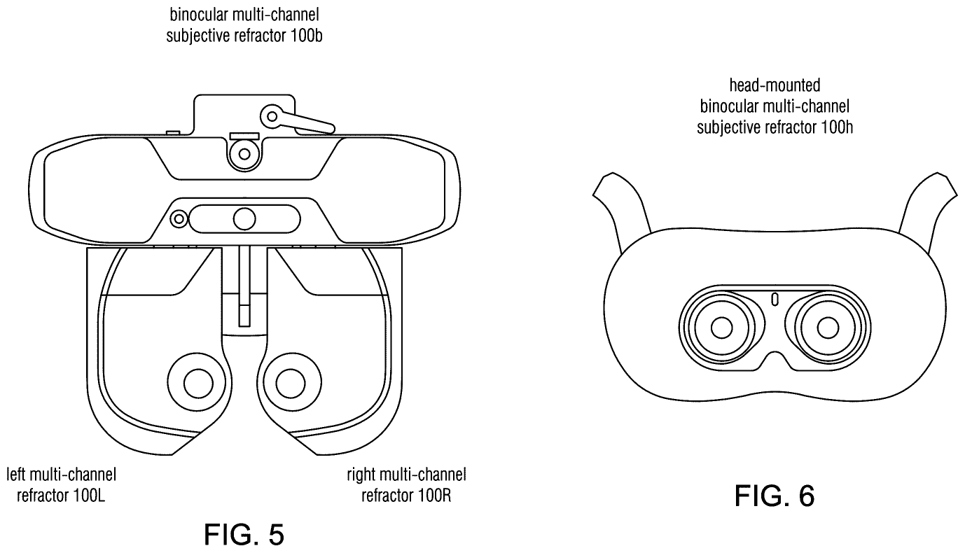

FIG. 5 shows that some embodiments of the multi-channel subjective refractor 100 can provide a refractor for each eye, thereby forming a binocular multi-channel subjective refractor 100b. Each of the individual multi-channel subjective refractors 100L and 100R for the left and right eyes, respectively, of this binocular refractor 100b can use or employ any of the previously described embodiments of the multi-channel subjective refractor 100.

FIG. 6 further shows that such a binocular multi-channel subjective refractor 100b can be implemented as a head-mounted binocular multi-channel subjective refractor 100h, which, again, can utilize any previously described embodiment of the individual multi-channel subjective refractor 100.

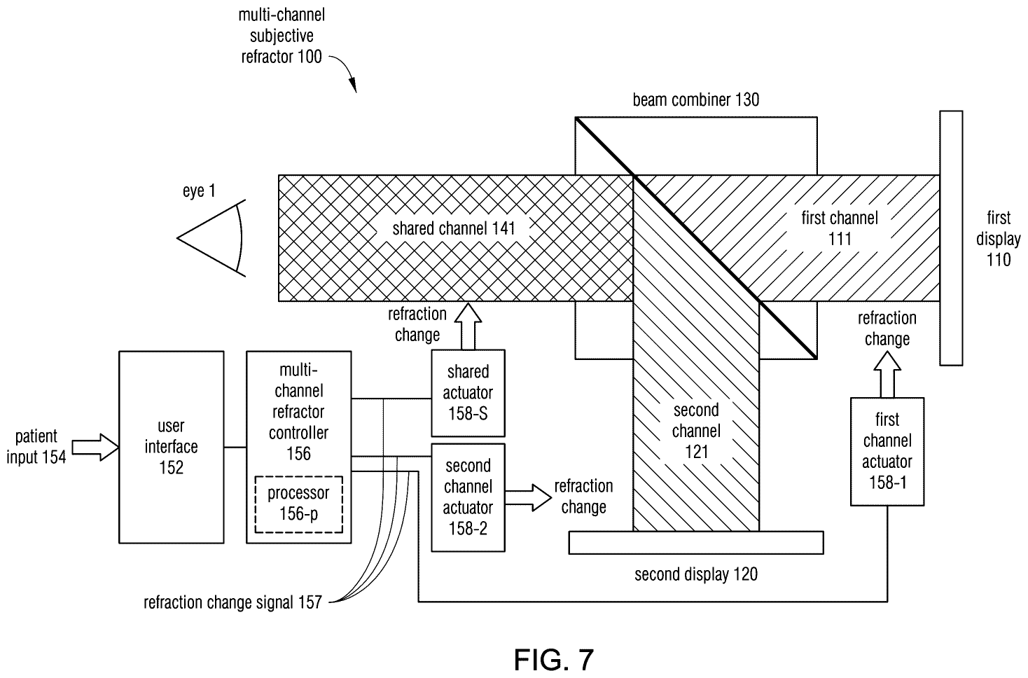

FIG. 7 shows that the operation of the multi-channel subjective refractor 100 can be partially or fully automated. A few, or several, or all steps of the operation of such automated refractors 100 can be automated, and therefore can be carried out by a lesser trained technician, or possibly even by the patient herself. Naturally, such automation can further reduce the chair time, or demand on the optometrist's lane, and thus can have multiple beneficial effects on the optometry practice. Such automated refractors 100 enable the patient herself to explore the refraction parameter space away from the chair or lane, in a preparation or waiting area. This allows the patient to take all the time needed to repeatedly try out various refractions, possibly for longer periods and allow her eyes to accommodate to the presented refractions. Such unhurried, self-guided refraction measurements with the multi-channel subjective refractor 100 may increase the accuracy and reliability of the eventually chosen optimal refraction.

Some embodiments of such an automated multi-channel subjective refractor 100 may include an automated refraction control system to receive an input from the patient; and to change at least one of the first refraction 113 and the second refraction 123, in response to the patient input. In more detail, the multi-channel subjective refractor 100 may include a user interface 152 to receive a patient input 154; a multi-channel subjective refractor controller 156, to generate a refraction change signal 157 in response to the patient input 154; at least two of a shared actuator 158-S, a first channel actuator 158-1 and a second channel actuator 158-2, coupled to the multi-channel subjective refractor controller 156, to change a corresponding refraction in response to the refraction modification signal 157.

In an example of the operation, the multi-channel controller 100 may present the letter E with a first refraction 113 of 2.0 D, and with a second refraction 123 of 2.5 D for an eye 1, as shown in FIG. 4B, side by side, and the patient may be prompted to create a patient input 154 by indicating which letter is sharper. Having received this patient input 154, the multichannel refractor controller 156, or simply refractor controller 156, can generate a refraction change signal 157 to increase or to decrease the first or second refraction 113/123 presented to the patient.

The patient can generate the patient input 154 in many different ways. The patient input 154 can be verbal, i.e. saying out loud which is the sharper image, and the corresponding electric control input can be generated by the optometrist, or a technician, by selecting a refraction change on a graphical user interface of the user interface 152. In these semi-automated designs, the role of the refractor controller 156 is only to generate a refraction change signal 157 that corresponds to the entered refraction change. In other semi-automated embodiments, the user interface 152 can have dials, knobs, sliders, or any other types of input devices, and the patient can be prompted to turn a knob to create a refraction change command for the refractor controller 156. In this case, the refractor controller 156 again only generates a refraction change signal 157 that corresponds to the refraction change command by the patient, to make the actuators 158-S, 158-1 and 158-2 to carry out the corresponding refraction changes.

In other, fully automated designs, the user interface 152 can be a voice recognition system that understands the verbal patient input 154 and translates it for the refractor controller 156. The patient input can be only which image 110i or 120i appeared sharper for the patient, but may not specify a refraction change value. In these designs, the refractor controller 156 can have a (search) algorithm installed in a processor, or computer, 156-p that actually determined what refraction change is most reasonable given the patient input 154.

In any of the above embodiments, the user interface 152 can be one of a graphic user interface, a voice recognition and control interface, a motion sensor, and an at least partially mechanical interface, including one of a slider, a knob, or a switch.

As discussed, the automated multi-channel subjective refractor 100 can be partially or fully automated. In non-automated traditional refractors, the refraction change is typically created by mechanical changes, e.g., by moving lenses in or out of the optical pathway mechanically.

In partially (semi-) automated multi-channel subjective refractors 100, the refraction change can be partially electronic. For example, the user interface 152 can be a voice recognition system, and the refractor controller 156 is tasked to convert the voice-recognized command into the electronic refraction change signals 157. These refraction change signals 157 eventually can actuate an electromotor that causes the translation of the first or second displays 110/120 along their translational stages 115/125, or move the movable lenses 114/124/144, or rotate the lenses of the stokes lens 116/126/146.

In some embodiments, the multi-channel subjective refractor 100 can be fully automated by including the optional processor 156-p, to scan a relevant refraction parameter space efficiently by executing a search algorithm; and to change the corresponding refraction according to the search algorithm. The refraction parameter space can include at least three dimensions: the defocus/sphere/optical power, the magnitude of the cylinder, and the direction of the cylinder. The latter two can be reparametrized as the cylinder in the 0 degree and in the 45 degree direction, or in the x and y direction. In more complex embodiments of the multi-channel subjective refractor 100, the refraction search space can be spanned by more than three dimensions.

Including the processor 156-p can be very useful, because the mere patient input 154 that, e.g., “the first image 110i with the first refraction 113 appeared sharper than the second image 120i with the second refraction 123”, does not determine unequivocally how to change the refraction. There can be more than one refraction changes that are well-motivated by the same patient input 154. As it is well known, naïve “brute force” scanning search algorithms can be very inefficient. There are now textbooks dedicated to how to accelerate and to improve the efficiency of search algorithms. The search algorithm, coded and stored in a computer-readable medium, in conjunction with the processor 156-p can be any of these optimization algorithms, such as convex optimization, conjugate gradient methods, genetic codes, neural network codes, linear, binary, hashing algorithms, artificial-intelligence-based algorithms, or any other search algorithm. Such search algorithms can change two or even three refraction parameters simultaneously, which enable search process on the multi-channel subjective refractor 100 to converge to the optimal refraction much faster than the naïve scanning methods.

In some embodiments, the multi-channel subjective refractor 100 can also potentially present a series of letters at different resolutions at the end of the refraction process to the patient to read off (or type) using the determined refraction, in order to get another measure of the patient's best corrected visual acuity.

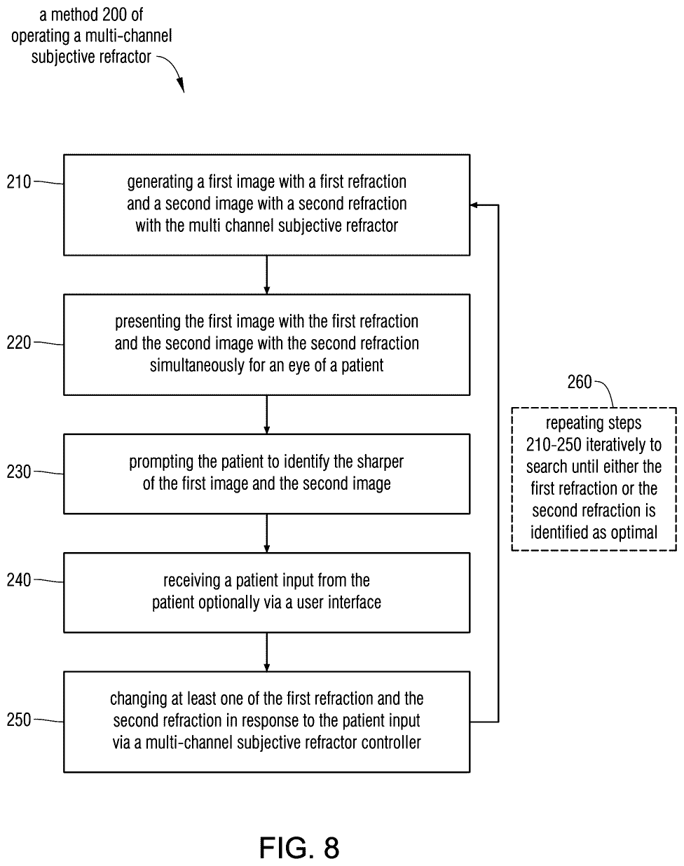

FIG. 8 shows a method 200 of operating the multi-channel subjective refractor 100. The method 200 can include the steps of:
generating 210 a first image 110i with a first refraction 113 and a second image 120i with a second refraction 123 with the multi-channel subjective refractor 100;
presenting 220 the first image 110i with the first refraction 113 and the second image 120i with the second refraction 123 simultaneously for an eye 1 of a patient by the multi-channel subjective refractor 100; and
prompting 230 the patient to identify the sharper of the first image 110i and the second image 120i.

This description of the method steps already captures a distinguishing factor of the refractor 100 and the method 200: that the first and second images 110i and 120i are presented in parallel, simultaneously for the patient.

The generating step 210 and the presenting step 220 can be carried out by the various embodiments of the multi-channel subjective refractor 100, as described in FIGS. 1-7 above. The prompting step 230 can be carried out either verbally by a medical technician or a doctor, or in an automated fashion, e.g. by a voice command generated by the multi-channel subjective refractor 100 itself. As is reasonable, in either of these cases, the patient should be given the necessary time to make a considered choice.

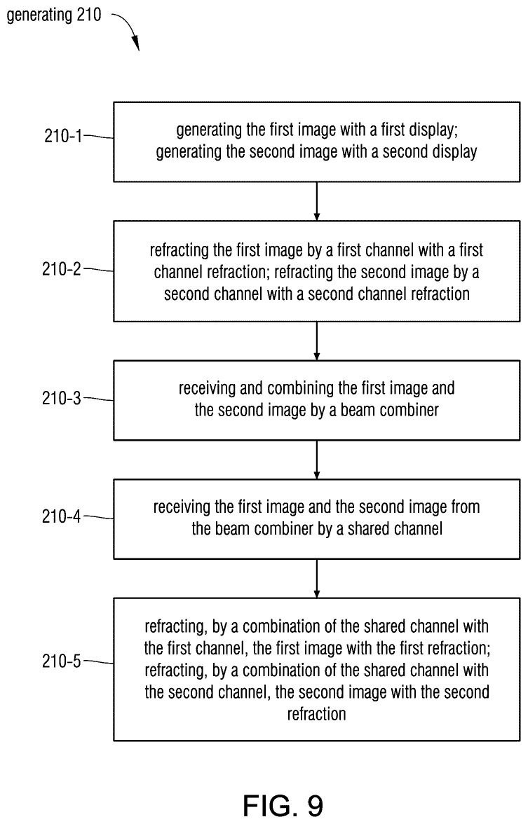

FIG. 9 shows in some detail that in the method 200 the generating 210 can comprise generating 210-1 the first image 110i with a first display 110; generating the second image 120i with a second display 120; refracting 210-2 the first image 110i by a first channel 111 with a first channel refraction 112; refracting the second image 120i by a second channel 123 with a second channel refraction 112; receiving 210-3 and combining the first image 110i and the second image 120i by a beam combiner 130; receiving 210-4 the first image 110i and the second image 120i from the beam combiner 130 by a shared channel 141; refracting 210-5, by a combination of the shared channel 141 with the first channel 111, the first image 110i with the first refraction 113; refracting, by a combination of the shared channel 141 with the second channel 121, the second image 120i with the second refraction 123; and presenting 220 the first image 110i with the first refraction 113 and the second image 120i with the second refraction 123 to an eye 1 simultaneously.

In some embodiments of the method 200 the first channel refraction 112 comprises at least one of a first channel defocus and a first channel cylinder; the second channel refraction 122 comprises at least one of a second channel defocus and a second channel cylinder; the first refraction 113 comprises at least one of a first defocus and a first cylinder; and the second refraction 123 comprises at least one of a second defocus and a second cylinder.

Some embodiments of the method 200 are related to FIGS. 2A-B and can comprise refracting the first image 110i by the first channel 111 with the first defocus and the first cylinder; and refracting the second image 120i by the second channel 121 with the second defocus and the second cylinder; without refracting the first image 110i and the second image 120i by the shared channel 141. In these methods 200, the first channel 111 can comprise at least one of a first movable lens 114, a first variable power lens, a first deformable mirror, a first phase modulator, and a first translational stage 115 to move the first display 110, to refract the first image 110i with the first defocus; and a first stokes lens 116, to refract the first image with the first cylinder; and the second channel 121 can comprise at least one of a second movable lens 124, a second variable power lens, a second deformable mirror, a second phase modulator, and a second translational stage 125 to move the second display 120, to refract the second image 120i with the second defocus; and a second stokes lens 126, to refract the second image 120i with the second cylinder. The relationships between the first/second channel defocus and cylinder and the first/second defocus and cylinder are shown in the refraction table in FIG. 2B and above.

FIGS. 3A-F show that other embodiments of the method 200 can comprise refracting the first image 110i and the second image 120i by the shared channel 141 with a shared refraction 143 that includes at least one of a shared defocus SD and a shared cylinder SC; wherein the shared channel 141 comprises at least one of a shared movable lens 144, a shared variable power lens, a shared deformable mirror, or a shared phase modulator, to refract the first image 110i and the second image 120i with the shared defocus SD; and a shared stokes lens 146, to refract the first image 110i and the second image 120i with the shared cylinder SC. These embodiments of the method 200 can further comprise the following.
(1) Refracting the first image 110i by the first channel 111 with the first channel defocus CD1 that combines with the shared defocus SD to create the first defocus D1, wherein the first channel 111 comprises at least one of a first movable lens 114, a first variable power lens, a first deformable mirror, a first phase modulator, or a first translational stage 115 to move the first display 110; and
(2) Refracting the first image 110i by the first channel 111 with the first channel cylinder CC1 that combines with the shared cylinder SC to create the first cylinder C1 using a first stokes lens 116; or
(3) Refracting the second image 120i by the second channel 121 with the second channel defocus CD2 that combines with the shared defocus SD to create the second defocus D2, wherein the second channel 121 comprises at least one of a second movable lens 124, a second variable power lens, a second deformable mirror, a second phase modulator, or a second translational stage 125 to move the second display 120; and
(4) Refracting the second image 120i by the second channel 121 with the second channel cylinder CC2 that combines with the shared cylinder SC to create the second cylinder C2, using a second stokes lens 126.

In some embodiments of the method 200 one of the refracting by the first channel 111 and refracting by the second channel 121 may not take place. In these embodiments, the shared defocus SD and the shared cylinder SC, refracted by the shared channel 141 is in fact the entire refraction of the channel which has no channel refraction.

As discussed before, one of the advantages of using the method 200 to operate the multi-channel subjective refractor 100 is that in this method the wide range of refractions can be implemented only in the shared channel 141, and the individual channels 111 and 121 may be used only to refract the corresponding images by a small, differential amount. Accordingly, in some embodiments, refracting by one of the channels may include refracting by less than two diopters in at least one of the first channel defocus CD1, the first channel cylinder CC1, the second channel defocus CD2 and the second channel cylinder CC2.

In some embodiments, the numerous degrees of freedom of the multi-channel subjective refractor 100 enable the method 200 for independently adjusting the first refraction 113 and the second refraction 123 by the first channel 111, the second channel 121 and the shared channel 141.

As FIGS. 4A-B show, an important aspect of the multi-channel subjective refractor 100 and the method 200 is that the latter includes presenting the first image 110i with the first refraction 113 and the second image 120i with the second refraction 123 simultaneously in one of an up-down image pair, a side-by-side image pair, and an image pair. In some embodiment, the method 200 can further include presenting one or more additional images with corresponding refractions simultaneously for the eye using one or more additional channels.

FIGS. 5-6 show that some embodiments can have a multi-channel subjective refractor 100 for each eye, thereby forming a binocular multi-channel subjective refractor 100b. This binocular multi-channel subjective refractor can be implemented as a head-mounted binocular multi-channel subjective refractor 100h.

FIG. 7 shows that some embodiments of the method 200 can further comprise receiving 240 the patient input 154 from the patient via the user interface 152; and changing 250 at least one of the first refraction 113 and the second refraction 123 in response to the patient input 154 via a multi-channel subjective refractor controller 100. The receiving 240 can include receiving the patient input 154 via the user interface 152 directly from the patient, or indirectly by the patient conveying the input to a medical professional for entering it into the user interface 152. As discussed above, in some exemplary indirect embodiments, the patient can identify verbally which image and refraction is sharper, and a medical technician, or the optometrist OD can enter the patient input 154 into the user interface 152. In direct embodiments the user interface 152 can include a voice command unit that recognizes the statement by the patient. Or there can be an electro-mechanical button, switch, slider, or knob at the hand of the patient, to select the sharper image and possibly, to command the multi-channel subjective refractor 100 to increase or decrease parameters of the refraction, such as to increase the defocus, or optical power of the refraction.

In some embodiments, the changing 250 can include generating a refraction change signal 157 by the multi-channel subjective refractor controller 156 in response to the patient input 154; and changing at least one of the first refraction 113 and the second refraction 123 in response to the refraction change signal 157 by at least one of the shared actuator 158-S, the first channel actuator 158-1 and the second channel actuator 158-2, coupled to the multi-channel subjective refractor controller 156.

In some embodiments, the method 200 can be semi-automated, wherein the patient 154 input includes a new refraction value for at least one of the first refraction 113 and the second refraction 123; and the generating the refraction change signal 157 includes translating the entered new refraction value into the refraction change signal 157 by the refraction controller 156.

Other embodiments of the method 200 can be automated, wherein the patient input 154 does not include a new refraction value for at least one of the first refraction 113 and the second refraction 123; the multi-channel refraction controller 156 comprises a processor 156-p; and the generating the refraction change signal 157 includes generating the refraction change signal 157 by performing a search algorithm to identify a new refraction value from the patient input by the processor 156-p.

FIG. 8 shows that the method 200 typically includes repeating 260 the generating 210, the presenting 220, the prompting 230, the receiving 240, and the changing 250 steps iteratively until either the first refraction 113 or the second refraction 123 is identified as optimal. This iterative process is much faster, more precise, and inflicts less mental fatigue with the here-described parallel-presenting multi-channel subjective refractor 200 than with traditional, sequentially-presenting refractors.

Some embodiments of the method 200 eliminate one more potential source of subjective bias by adding a step of exchanging a spatial presentation of the first image 110i with the first refraction 113 and the second image 120i with the second refraction 123 after the optimal refraction has been identified; and testing whether the optimal refraction remained optimal after the exchange. In an example, if at the end of the iterative search process the patient states that the first image 110i, shown with the first refraction 113 spatially above the second image 120i is the optimal, then the two images and refractions can be exchanged, followed by re-presenting the first image 110i with the first refraction 113 spatially below the second image 120i with the second refraction 123. A possible way to do it is to exchange both the images and the refractions, making the first image 110i the second image 120i, and the first refraction 113 the second refraction 123, and vice versa, and then prompt the patient to again identify the optimal refraction. Other, equivalent designs can achieve the same verification or confirmation.

Referring back to the duochrome tests, in some embodiments of the method 200 the generating 210 can comprise generating the first image 110i and the second image 120i with approximately the same background color.

In some embodiments of the method 200, the generating 210 can comprise generating the first image 110i with the first refraction 113 and the second image 120i with the second refraction 123 that is different from the first refraction 113.

Finally, it is mentioned that for patients who have more accommodation in their eyes, the method 200 can be used with appropriate adjustments.

### Autorefractor

The above-described embodiments of the multi-channel subjective refractor 100 are capable of determining a patient's refraction very efficiently by an iterative search process that is guided by the subjective feedback of the patient. Naturally though, the search process converges faster the closer it starts to the eventual refraction. Therefore, the operation of the multi-channel subjective refractor 100 can be accelerated and made to converge faster, if it is combined with an objective refractor, or autorefractor, that is configured to determine the refraction of the patient approximately before starting the subjective method 200. Then the subjective method 200, described in relation to FIGS. 8-9, can be started from this approximate refraction, determined by objective methods. Such objectively determined refractions typically approximate the best refraction within less than a diopter. Starting the method 200 so close to the best refraction greatly accelerates the method 200 and increases its precision.

To address the above-articulated need, an objective refractor 300, or autorefractor 300 has been developed, and described here. Over the years, many autorefractors have been proposed and developed. The uniqueness and inventiveness of the here-described autorefractor 300 derives at least from the following. (1) The structure of the autorefractor 300 is particularly simple, because it is based on a notable physical insight regarding the forward and backward light propagation through adjustable optical systems. (2) The autorefractor 300 is uniquely well-suited to be combined with the multi-channel subjective refractor 100 because the autorefractor 300 can share optical elements with the multi-channel subjective refractor 100.

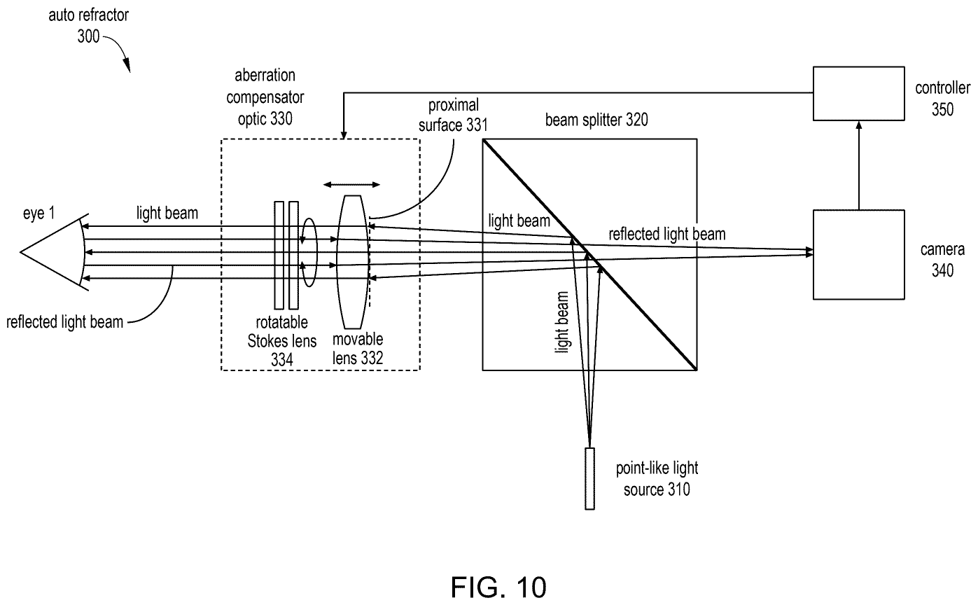

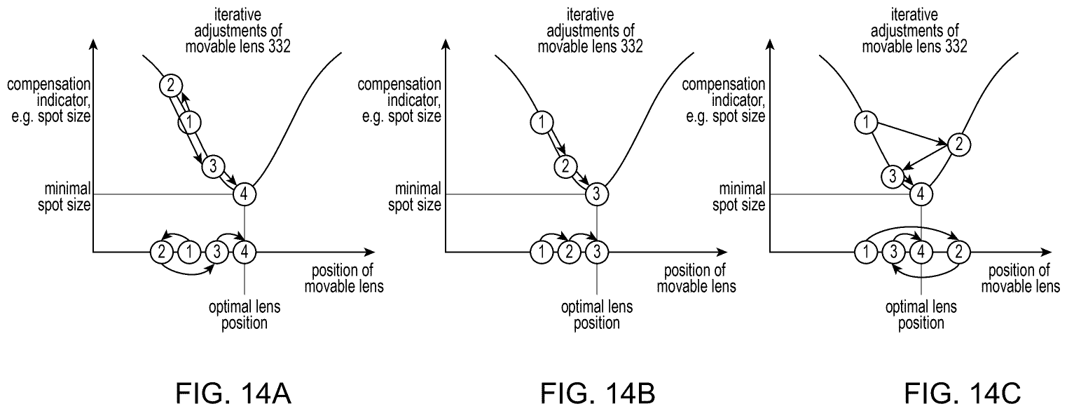

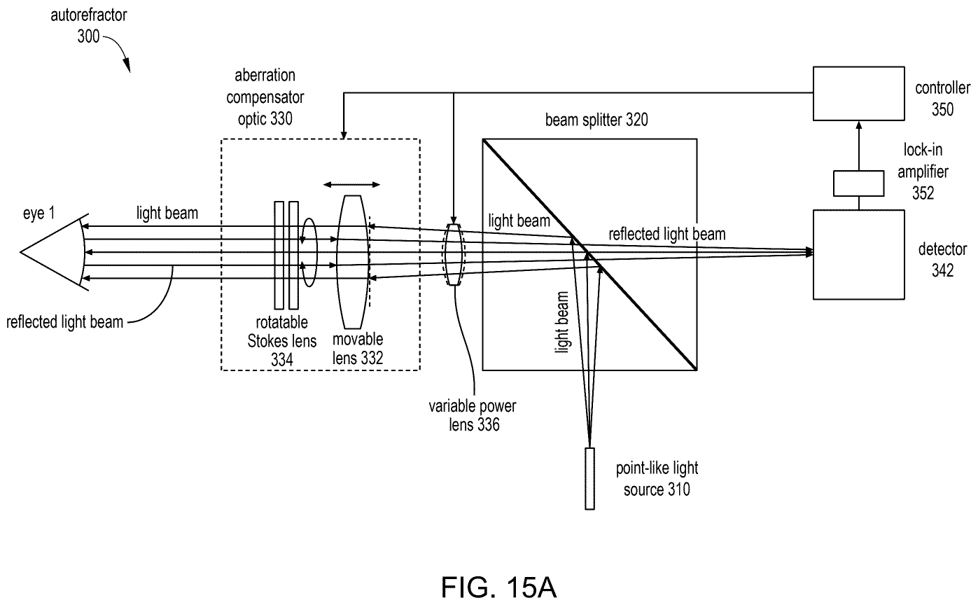

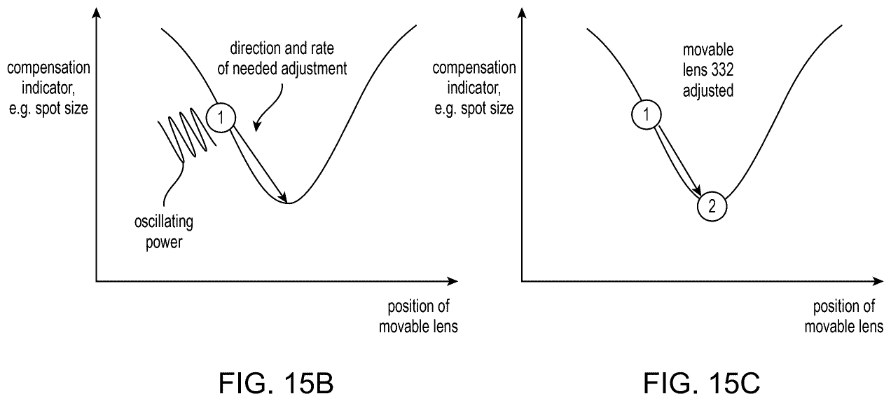

While in some embodiments the autorefractor 300 can be advantageously combined with the multi-channel subjective refractor 100, it can be also used as a self-standing refractor. For the sake of clarity, FIGS. 10-13B describe the autorefractor 300 by itself. Then, FIGS. 14A-D and FIG. 15 describe combinations of the autorefractor 300 with the multi-channel subjective refractor 100.

FIG. 10 shows an embodiment of the autorefractor 300 that includes a point-like light source 310, to emit a light beam; a beam splitter 320, to direct the light beam; an aberration compensator optic 330, to receive the light beam from the beam splitter 320, to propagate the light beam with a compensating aberration to an eye 1 of a patient, and to propagate a reflected light beam, reflected by the eye 1, to the beam splitter 320; wherein the beam splitter 320 is configured to direct the reflected light beam, received from the aberration compensator optic 330; a camera 340, to receive the reflected light beam from the beam splitter 320, and to capture an image formed from the reflected light beam; and a controller 350, to determine a compensation indicator of the reflected light beam from the captured image, and to adjust the aberration compensator optic 330 to improve the compensation indicator. The compensating aberration, imparted by the aberration compensator optic 330, can be a compensating defocus, a compensating astigmatism, or a combination of the two, or any of the above combined with a compensated higher order aberration. For other embodiments, see below.

Broadly, an operating principle of the autorefractor 300 can be understood starting with the insight that if the optical system of the eye, whose main elements are the cornea and the lens, works optimally, then the eye images a (distant) point-like light source onto an essentially point-like image spot on the retina (“retinal spot”), whose diameter is limited only by diffraction. (Here the “lens” can be the natural crystalline lens, or an implanted intraocular lens (IOL) or Light Adjustable Lens (LAL)). If then this essentially point-like retinal spot is imaged by a camera, positioned into a conjugate plane of the retina, through the same optical system of the eye, then the point-like retinal spot will be imaged as a minimal-sized image spot in the camera 340, whose size is again limited only by diffraction.

However, often the optical system of the eye is not optimal. It has low order aberrations like defocus and cylinder, and higher order aberrations like spherical aberrations. If the aberration of the optical system of the eye is a defocus, then a point-like light source is imaged as a larger diameter spot on the retina. If the aberration of the optical system is a cylinder, or toric, aberration, then a point-like source is imaged as an elongated, or elliptical spot on the retina. If then these already extended retinal spots are imaged by the camera 340, placed into a conjugate plane of the retina, they generate an even more extended image spot in the camera 340. Since the increased size and the non-circular shape of the image spot are caused by the aberrations of the eye, therefore they can be used as an indicator of these aberrations of the eye. This dependence of the size and shape of the imaged spot on the aberration of the optical system of the eye is utilized as an operating principle of the autorefractor 300.

Broadly, the autorefractor 300 builds on this insight by employing an adjustable aberration compensator optic 330 to compensate the aberration of the eye. In operation, the aberration compensator optic 330 is adjusted to minimize the imaged spot in the camera 340. At this setting, the aberration compensator optic 330 optimally compensates the aberrations of the eye. This optimally compensating setting can be used to determine the aberrations of the eye.

In more detail, an operating principle of the autorefractor 300 is that if the aberration compensator optic 330 compensates the aberrations of the optical system of the eye optimally, then the point-like light source 310 will be imaged on the retina as an optimally small retinal spot. Subsequently, the light reflected from the small retinal spot propagates through the optical system of the eye and the aberration compensator optic 330 for a second time, and forms an optimally small image spot in the camera 340. But as soon as the aberration compensator optic 330 does not compensate the aberration of the eye optimally, the size of the retinal spot increases, and therefore the size of the image spot in the camera 340 increases even more. The increase of the size of the image spot from the optimal, or minimal size can be used as an indicator of how far the aberration compensator optic 330 is from compensating the aberration of the eye 1 of the patient. In this sense, the deviation of the size of the image spot from a minimal diameter is an “aberration compensation indicator”, or “compensation indicator” for brevity. The compensatory aberration of the aberration compensator optic 330 that minimized this compensation indicator can be referred to as the optimal compensatory aberration. When the compensation indicator is optimal, e.g., the image spot size is minimal, then the refractive properties of the aberration compensator optic 330, such as its defocus and cylinder, can be used as the prescription for the patient. Spectacles prescribed with this optimal compensatory aberration will eliminate, or at least minimize, the aberration experienced by the patient wearing these prescribed spectacles.

In more technical terms, the image of a light propagating through an optical system can be constructed as a convolution of the source with the point spread function (PSF) of the optical system. If the autorefractor 300 is adjusted to optimally compensate the defocus, and in general the aberrations, of the eye of the patient, and the point-like light source 310 is placed into the conjugate plane of the retina, then the image of the point-like light source 310 on the retina, constructed by a convolution with the PSF of the combined optical system of the eye and the autorefractor 300, will be an optimally small spot, it's size limited primarily by diffraction. If the autorefractor 300 is not adjusted to compensate the defocus and aberrations of the patient, then the spot size on the retina retinal spot will be larger.

Subsequently, the light reflected from this retinal spot again propagates through the same combined optical system, now in reverse, eventually imaged by the camera 340. Accordingly, the image in the camera 340 is generated by a second convolution, now of the retinal spot again with the PSF of the combined optical system of the eye and the auto refactor 300. If the autorefractor 300 is adjusted to compensate the defocus and aberrations of the patient, and the camera 340 is placed in a conjugate plane of the retina, then the size of the image spot in the camera 340 will be minimal, limited primarily by diffraction. As before, however, if the autorefractor 300 is not adjusted to compensate the defocus and aberrations of the patient optimally, then the backward convolution of the retinal spot with the combined eye plus autorefractor 330 PSF again increases the image spot size in the camera 340, just like the forward convolution did. In short, if the autorefractor 300 is not adjusted to compensate the defocus and aberrations of the patient optimally, then both the forward propagation and the backward propagation of the light increases the spot size, thereby generating an image spot in the camera 340 noticeably larger than the minimal spot size. As described earlier, the autorefractor 300 uses this deviation of the image spot size from its minimal value as a “compensation indicator”. The aberration compensator optic 330 is adjusted until it minimizes this compensation indicator. The settings of the aberration compensator optic 330 at this minimum of the compensation indicator can be then identified as the optimal compensatory aberration. In stand-alone implementations of the autorefractor 300, this optimal compensatory aberration can be used as the spectacle prescription for the patient. In combined implementations of the autorefractor 300 with the multi-channel subjective refractor 100, this “optimal compensatory aberration” can be used by the multi-channel subjective refractor 100 as a suggested starting point for the patient to the method 200 of operating the multi-channel subjective refractor 100.

In some embodiments, the point-like light source 310 refers to a light source with a small but finite diameter. This diameter of the point-like light source 310 can be less than 2 mm, 1 mm, or 0.5 mm. In some cases, this diameter can be in a range of 0.2 mm-0.4 mm. The point-like light source 310 can be a tip of an optical fiber, a laser, a luminous disk, an LED, or an LED with a pinhole in front of it.

In self-standing embodiments, the autorefractor 300 can operate with a wide range of wavelengths in the visible part of the spectrum that extends from about 400 nm to an upper limit in the 700 nm-750 nm range. Or, it can operate in the infrared part of the spectrum that extends from about 700 nm-750 nm to an upper limit in the range of 100 microns-1,000 microns. These ranges are approximate and depend on the various definitions. In embodiments, where the autorefractor 300 is combined with the multi-channel subjective refractor 100, it can be useful to utilize a light with a wavelength longer than the one used by the multi-channel subjective refractor 100. In such combined embodiments, the point-like light source 310 can emit the light beam as an infrared light with a wavelength exceeding 650 nm, 700 nm, 750 nm, or 800 nm, up to an upper limit, which again can be in the range of 100 microns-1,000 microns. In some embodiments, the light of the point-like light source 310 can be in the near infrared, or near-IR range.

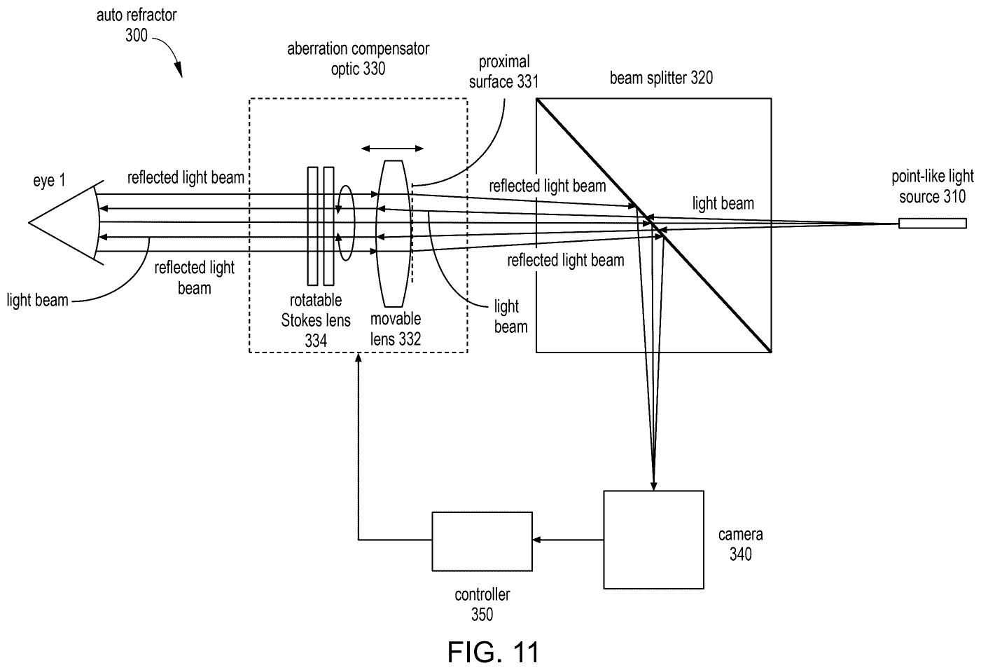

FIG. 10 and FIG. 11 show two practical embodiments of the autorefractor 300, which are somewhat complementary to each other. In the design of FIG. 10, the beam splitter 320 can be configured to redirect the light beam from the point-like light source 310 toward the aberration compensator optic 330, on its way to the eye 1; and to transmit the reflected light beam from the eye to the camera 340. This functionality can be achieved by using a partially transmitting mirror in the beam splitter 320 in some designs. However, in such designs the signal-to-noise ratio is rather poor, because the back-reflected light greatly boosts the background noise. To address this problem, in some embodiments the beam splitter (BS) 320 can be a polarizing beam splitter (PBS) 320; and the point-like light source 310 can be configured to emit the light with an s-wave polarization, defined relative to a diagonal surface of the polarizing beam splitter 320. A widely used description of the polarization directions is to define the direction of propagation of the light beam by the vector w, and the normal vector of the diagonal surface of the polarized beam splitter 320 by n. With this preparation, the s-wave polarization direction is parallel with the vector product of w and n: s|w×n. In the embodiment of FIG. 10, if the point-like light source 310 emits light with a such-defined s-wave polarization, then the polarized beam splitter 320 will operate as described: reflect the light from the point-like light source 310 toward the eye 1, while simply transmitting a substantial portion of the reflected light beam, reflected from the eye 1, so that the camera 340 can image the reflected light beam. This is because the retina reflects the light without a preferred polarization direction. As a simple estimate, it is reasonable to characterize the light reflected by the retina as depolarized. The polarized beam splitter 320 will transmit about 50% of such a depolarized light. The camera 340 can be also called a camera sensor 340.

FIG. 11 shows a complementary embodiment. In this design of the autorefractor 300 the beam splitter 320 can be configured to transmit the light beam from the point-like light source 310 toward the aberration compensator optic 330, on its way to the eye 1; and to redirect the reflected light beam from the eye 1 to the camera 340. As before, in an embodiment this functionality can be realized by utilizing a beam splitter BS 320 that is a polarizing beam splitter PBS 320; but by configuring the point-like light source 310 to emit the light with a p-wave polarization, defined relative to a diagonal surface of the beam splitter 320. Recalling the above definition of s-wave polarization, the p-wave polarization is customarily defined as a vector product of the propagation direction vector w and the s polarization direction. In formula: p|w×s.

In embodiments of the autorefractor 300, the aberration compensator optic 330 can be configured to impart a compensating defocus by including at least one of a movable lens 332, slidably movable by a translation stage; a movable lens group, wherein at least one lens of the lens group is slidably movable by a translation stage; a variable power lens that is variable by mechanical, fluidic, or electro-optical means; and a variable power reflective optic. FIGS. 10-11 show embodiments where the aberration compensator optic 330 includes a movable lens 332.

In some autorefractors 300, the aberration compensator optic 300 can be configured to impart a compensating astigmatism by including a rotatable Stokes lens pair 334. Astigmatism is interchangeably also called a cylinder, or a toric aberration. Again, there can be several other embodiments that compensate the astigmatism by other designs.

As noted above, the operating principle of the autorefractor 300 involves that the optical path from the point-like light source 310 to the retina is essentially the same as the optical path from the retina to the camera 340. This design concept can be captured via the optical elements of the autorefractor 300 itself, e.g., by requiring that the optical path lengths from a proximal surface 331 of the aberration compensator optic 330 to the point-like light source 310 and to the camera 340 be the same, in some embodiments.

Returning to the discussion of the aberration compensation, in some embodiments the compensation indicator is a spot size in the image of the reflected light beam, captured by the camera 340. The controller 350 is configured to adjust the aberration compensator optic 330 to minimize this image spot size. This adjustment can include slidably moving the movable lens 332, or a lens of a movable lens group, or rotating the rotatable Stokes lens 334. In embodiments, the spot size is minimal when an aberration of the aberration compensator optic 330 compensates optimally an aberration the eye of the patient, within a tolerance. The tolerance can be impacted by many factors, e.g. by the step size of the position of the movable lens 332, the noise in the camera 340, the sharpness of the image spot, among others.

The optimum of the compensation can be determined by different methods. One such method is to move the movable lens 332, and to rotate the rotatable Stokes lens 334 to minimize the spot size, but then to continue the movement or rotation until the image spot size starts to visibly grow again. By tracking or plotting the spot size, the minimum of this spot-size curve can be determined, and the corresponding location of the optimal lens position and lens rotation angle can be read off. Such methods have different names, including undershoot-overshoot.

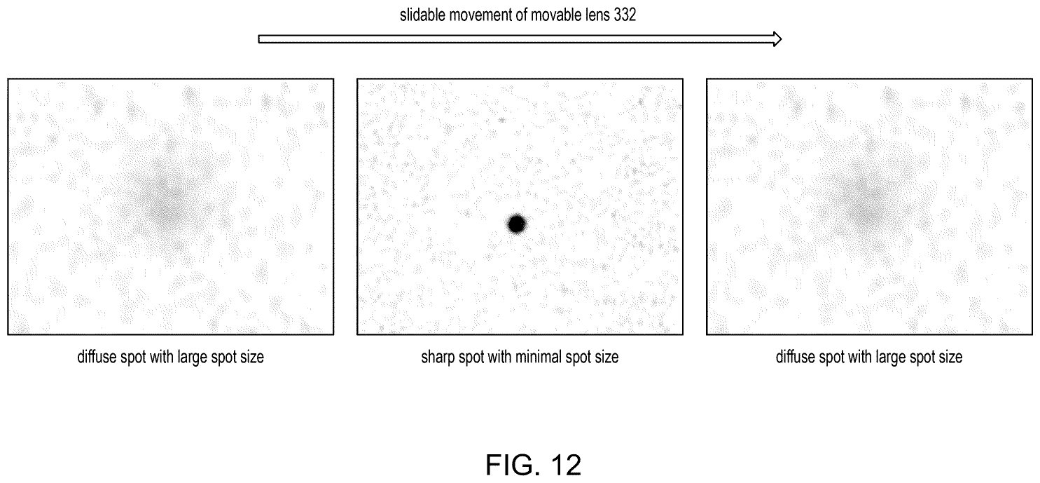

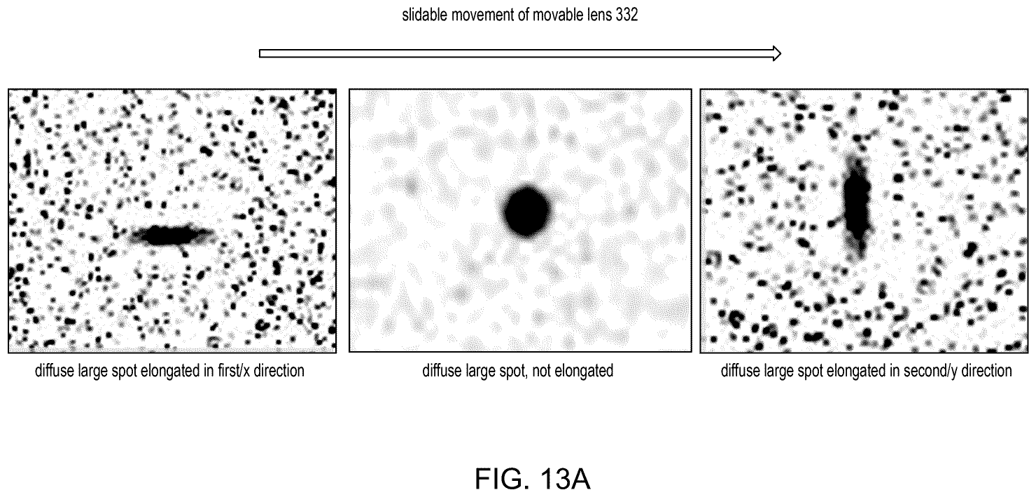

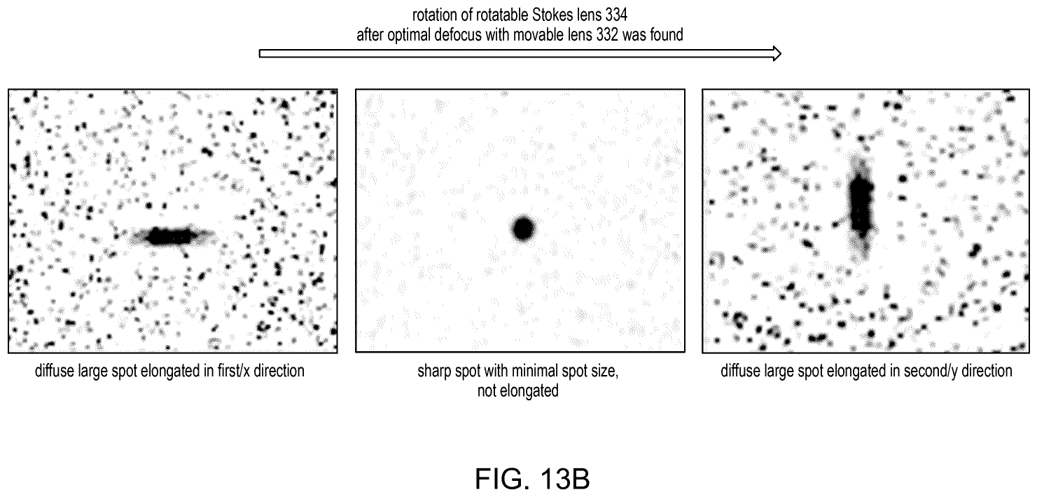

FIGS. 12 and 13A-B show exemplary image sequences for the controller 350 adjusting the aberration compensator optic 330. In FIG. 12, the controller 350 is slidably moving a movable lens 332. When the power, or defocus of the aberration compensator optic 330 undercompensates the power-deficiency of the eye, then the image of the returning light beam is a diffuse spot with a large diameter, as shown in the left panel. When the movable lens 332 is moved into the optimal position by the controller 350, then the image spot becomes sharp and has a small diameter, as shown in the center panel. When the spot is sharp and its size is minimal, that indicates that the aberration compensator optic 330 optimally compensated the power deficiency, or defocus of the eye 1. If the controller 350 then moves the movable lens 332 even further, then the aberration compensator optic 330 overcompensates the defocus, and the image again becomes a diffuse spot with a large spot size, as shown in the right panel. A short back-and-forth sliding protocol of the movable lens 332 enables the controller 350 to identify the optimal position of the movable lens 332, and from this to deduce the optimally compensating power, needed to be prescribed for the patient by the optometrist.

FIGS. 13A-B describe the analogous optimization, or search, process, this time also involving the rotatable Stokes lens 334. The three panels of FIG. 13A show the camera image spots of the retinal spot from the eye of a patient who has astigmatism, which was not yet compensated by the Stokes pair 334. The camera images were taken as the movable lens 332 was moved. Astigmatic optical systems focus the beam in a first plane along the z axis only in one direction. In the present case, the beam is focused in the y direction, but still unfocused in the x direction. Hence the image spot is narrow/thin in the y direction and wide/thick in the x direction, resulting in a spot, elongated in the x direction. The center panel shows that as the movable lens 332 is moved, the y-focusing gets worse, but the x-focusing gets better. This produces a large, somewhat diffuse image spot at the best compromise position along the z axis. Finally, the right panel shows that when the movable lens is moved further along the z axis, it reaches the position when the beam is best focused in the x direction, but is now poorly focused in the y direction. This produces an image spot elongated in the y direction.

FIG. 13B shows a sequence of camera images which document the search for an optimally compensating aberration by the aberration compensating optic 330, where the search is performed in a multidimensional space. Here the movable lens 332 is slid to find the optimally compensating defocus. Further, the Stokes lenses 334 are rotated to find the power and direction of the optimally compensating cylinder. The effective optimization in this three-dimensional parameter space can be performed by various algorithms. The simplest is the grid search. Beyond that any of the large number of more efficient searches can be performed, such as gradient searches, steepest descent searches, conjugate gradient searches, tree searches, genetic algorithms. Each of these search methods can be improved by training and implementing a machine learning layer.

In FIG. 13B, a one dimensional portion of the three dimensional search is shown. Here the optimal defocus compensation has been already achieved by sliding the movable lens 332. The three panels show the camera images as one of the Stokes lens 334 is rotated, in search of the optimal compensation of the astigmatism of the patient. In the first panel, the Stokes lens 334 undercompensates the cylinder in a first direction, and therefore the image spot is elongated along the first direction. In the center panel, the rotation of the Stokes lens 334 managed to optimally compensate the astigmatism. In the last panel, the further rotation of the Stokes lens 334 now overcompensates the astigmatism, resulting in the image spot getting elongated in a second direction. A difference relative to FIG. 13A is that at the optimal rotation angle in the center panel, both the defocus and the astigmatism have been optimally compensated. Therefore, the size of the image spot in the center panel is optimally small and its contours are sharp.

As before, rotating the Stokes lens 334 forward and backward a few times, possibly in conjunction with moving the movable lens 332 can be used to find the minimal spot size and thereby to identify the optimally compensating defocus and astigmatism. Also as before, the optimal compensating aberration of the aberration compensator optic 330 can be prescribed by an optometrist as a prescription of the patient. This procedure is sometimes also called determining the refraction of the patient, or simply “refracting the patient”.

To sum, the (compensating) aberration of the aberration compensator optic 330 includes a power, a cylinder, and an axis of the cylinder, and each of these optimally compensate a power, cylinder, and axis of the cylinder of the eye of the patient when the spot size of the image in the camera 340 is minimal, within a tolerance. The so-determined optimal compensating aberration can be used as a prescription for the patient.

It was mentioned above that some modifications, or corrections, may be needed to translate the optimal compensating aberration of the aberration compensator optic 330 into a prescription for the patient. One such modification arises from the fact that the refractive index of the eye varies with the wavelength of the light. Thus, in embodiments where the autorefractor 300 uses an infrared light, the power and cylinder that optimally compensates the aberration of the eye in the infrared is somewhat different from the optimally compensating values in the middle of the visible spectrum, such as around a wavelength around 550 nm. With this in mind, a prescription for the patient can be determined by correcting the optimally compensating aberration of the aberration corrector optic 330 for a dispersion of a refractive index of the eye of the patient by at least one of a defocus of the camera 340, a defocus of the point-like light source 310, a combination of these, or a software of the controller 350.

As discussed, in embodiments, the compensation indicator can be a size, or diameter, of the image spot. However, other embodiments are also possible, or can offer benefits. For example, the compensation indicator again can be related to the spot from the reflected light beam in the image captured by the camera 340, but the controller 350 can be configured to adjust the aberration compensator optic 330 for properties other than to minimize the spot size. Instead, the controller 350 can be configured to adjust the aberration compensator optic 330 to minimize an ellipticity of the spot, to sharpen a contour of the spot, or to maximize a brightness of the spot. Or, the controller 350 can monitor more than one properties and adjust the aberration compensator optic 330 to optimize a combination of the monitored properties.

In some embodiments, the aberration compensator optic 330 can be configured to also compensate a higher order aberration of the eye of the patient, within a tolerance. The embodiments of such aberration compensator optics 330 may involve more complex optical elements that can controllably vary higher order aberrations. Examples include deformable reflective elements, or fluid-filled lenses.

In any of the above embodiments, the controller 350 can be configured to improve the compensation indicator in an iterative manner, in order to eventually optimize the compensation indicator. For example, the controller 350 can slide the movable lens 332 past its optimum, note that the diameter of the spot started to increase, and then reverse the slide. The compensation indicator can be optimized with a few such back-and-forth iterations. A large number of search algorithms are known that reduce, or minimize, the number of steps needed to find the optimum, as mentioned before.

FIGS. 14A-C show one of the problems of the simple iterative search algorithms. In a first step, the movable lens 332 can be moved to a first position (1), as shown, and the compensation indicator, such as the spot size can be determined. Then, the movable lens 332 can be moved to position (2), and the spot size can be determined again. From here on the algorithm to find the smallest spot size branches out. FIG. 14A shows that if the spot size increased, then the movable lens 332 should be moved in the opposite direction. Probably the search will require at least two more steps (3) and (4) to find the optimum.

If the spot size decreased, then next it needs to be determined which side of the optimum position (2) is. FIG. 14B shows the case when position (2) is on the same side of the optimum as position (1). In this case, the movable lens 332 needs to be moved in the same direction to get to position (3). It is possible that the search will get very close to the optimum in this step (3). However, FIG. 14C shows the case when position (2) is on the opposite side of the optimum as position (1). If this case, the movable lens 332 needs to be moved in the opposite direction to a position (3), and in all likelihood, a step (4) will be needed to get very close to the optimum. If the movable lens 332 is moved in smaller steps, then the number of steps to reach the optimum increases. Some embodiments may not use fixed step sizes, but decrease the step size as the method progresses. This allows the method to “zoom in” on the optimum and find it with higher precision. Visibly, with decreasing step sizes and the corresponding increasing of the number of steps, the number of possible branches of the search process increases rapidly, making the control of the method more challenging. Also, this increasing number of steps takes up increasing amount of precious time in the optometrist office, and makes the patient impatient. Therefore, there is a need for improved embodiments of the autorefractor 300 that inform the search algorithm about the direction and size of the steps so that the time to find the optimum decreases.

FIGS. 15A-C show embodiments and algorithms that reduce the number of iterative steps needed to find the optimal compensation indicator, such as the minimal spot size. FIG. 15A shows the autorefractor 300 of FIG. 10 with an additional variable power lens 336 in it, for example, between the beam splitter 320 and the aberration compensator 330. Further, the previous camera 340 is just one embodiment of the presently shown detector 342. The optical power of the variable power lens 336 can be quickly oscillated. Oscillating the power of the variable power lens 336 oscillates the size of the spot detected by the detector 342. If increasing the power of the variable power lens 336 decreases the spot size, then in step (2) of the search method the movable lens 332 is moved to further increase the overall optical power of the system in order to move toward the optimum. If, on the other hand, increasing the power of the variable power lens 336 increases the spot size, then in step (2) the movable lens 332 is moved to decrease the overall optical power of the system in order to move toward the optimum. Put another way, the autorefractor 300 of FIG. 15A determines a direction and (optionally) a magnitude of the local gradient of the spot size vs. power curve and adjusts the movable lens 332 according to the determined direction and magnitude of the gradient. In more abstract terms, the variable lens 336 of the autorefractor 300 determines and provides local (gradient) information about the spot size that informs the global search and enables the autorefractor 300 to find and to converge to the optimum more efficiently.

FIGS. 15B-C illustrate the autorefractor 300 in operation. The optical power of the variable power lens 336 is oscillated around the initial value (1). The direction and magnitude of the induced change of the spot size is determined. The determined direction and magnitude are then used to inform the adjustment of the movable lens 332. Based on this informing and on previous modeling and calibration, the controller 350 adjusts the movable lens 332 to a new position (2) that it estimates most likely minimizes the spot size. Experience shows that in a large fraction of cases, already position (2) can be very close to the optimum, or minimum spot size. In the few cases position (2) does not reach the optimum, then repeating the above step is very likely to do so.

FIG. 15A shows some technical details of how to implement these principles. The phase and magnitude of the oscillating spot size is determined by the detector 342 as a function of time. Examples of the detector 342 include a fiber tip, which is configured to carry the detected light to a photodiode. An alternative embodiment is a pinhole with a photodetector behind it. In both cases, if the spot size of the incoming beam increases, then the light is distributed over a larger area and so the amount of light hitting the fiber tip or the pinhole decreases. Thus, the inverse of the amount of the detected light can be used as a measure of the spot size. These are fast embodiments of the detector 342. As mentioned, some embodiments may use a camera 340. In such embodiments, though, in every image the edge of the spot needs to be determined and then the size/diameter of the spot needs to be computed. This makes the camera 340 a slow, but possibly more accurate embodiment of the detector 342.

The detector 342 can send the signal carrying the amount of detected light intensity to a lock-in amplifier 352. The lock-in amplifier 352 filters out the magnitude and phase of the oscillating light intensity signal that has the same frequency as the oscillation of the variable power lens 336. This filtering determines both the direction and the magnitude of the spot size change as a function of the change of the optical power of the variable power lens 336. As an example, the sign of the detected and filtered light intensity carries the information whether the spot size increases or decreases with the increase of the optical power. In the phase language, the signal being negative, i.e. 180 degree out of phase indicates that increasing optical power decreases the spot size, as happens in positions (1) or (3) in FIG. 14C. In contrast, when the signal is positive, i.e. in phase indicates that increasing optical power increases the spot size, as happens in position (2) in FIG. 14C.

In different embodiments, varying the power of the variable power lens 336 can be realized in different ways. In principle, in some embodiments there is no need for a discrete variable power lens 336. Rather, the position of the movable lens 332 can be oscillated. However, creating such mechanical oscillations for larger lenses is challenging, as the movable lens 332 can be heavy, and creating mechanical oscillations may induce undesirable side effects. Therefore, some embodiments employ a separate variable power lens 336 that includes a liquid lens. The optical power of a liquid variable power lens 336 can be oscillated with a frequency of 100 Hz-5,000 Hz, in some typical lenses with a frequency of around 1 kHz. Other variable power embodiments can be constructed as well that use a small mechanically movable lens, a deformable lens, a deformable mirror, or an electroactive device.

In a variant of the variable power lens 336, the cylinder power, or toric astigmatism, of the variable power lens 336 can be varied as well. Varying the cylinder power and the cylinder axis of this variable cylinder power lens 336 informs the search algorithm that seeks the optimal cylinder power and axis by rotating the Stokes lenses 334. The cylinder power and the cylinder axis of this variable (cylinder) power lens 336 can be oscillated in a way analogous to the variable spherical power lens 336, described above in relation to FIGS. 15B-C.

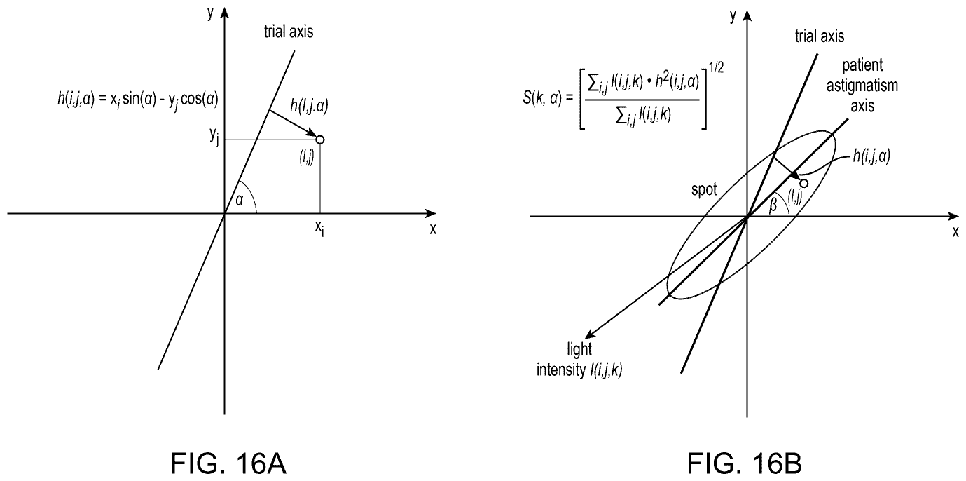

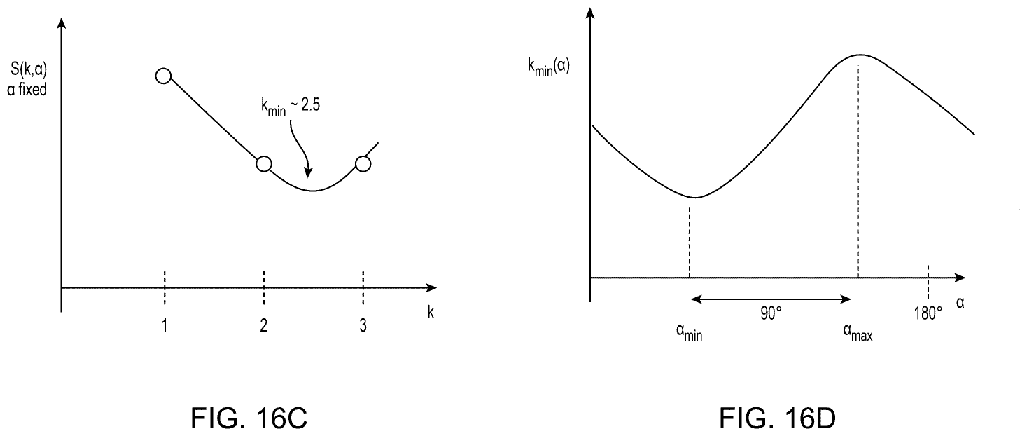

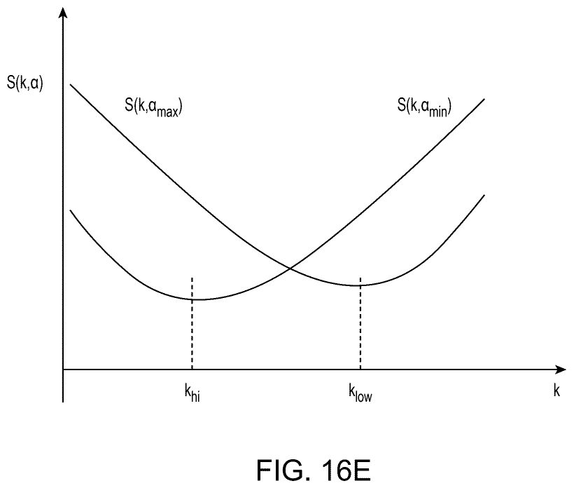

FIGS. 16A-E describe the operation of yet another embodiment of the autorefractor 300 which can deliver similar functionalities without utilizing an oscillating variable power lens 336. Instead, this embodiment of the autorefractor 300 sweeps through a relevant region of the optical power by moving the movable lens 332 of FIG. 10 along its axis of motion z, and captures the camera images at a sequence of positions. These positions, or stops, are often equidistant. An exemplary sequence is shown in FIG. 13A. The type of astigmatism shown is “on-axis”, also known as “J0” or the Z_{2,2} Zernike function. The sign of J0 is either positive or negative, depending on the sign of the direction of motion of the movable lens 332 relative to the patient's eye.

Moving the movable lens 332 without needing a variable power lens 336 is possible in these embodiments because the movable lens 332 is not oscillated at a high frequency. Rather, it is moved linearly in the z direction, and makes only a few stops. The number of stops can be in the range of 2-10, often in the range of 3-5. A sequence of the images is captured at these stops, and then the controller 350 analyzes the captured images and finds the minimal spot size from the analysis of this sequence of images, as described below in some detail.

FIG. 16A shows the construction of a reference frame to analyze the images of the camera 334. The (x,y) coordinates of a point in the image relative to the image center are discretized as (xᵢ,yⱼ), e.g. according to the corresponding image pixel indices (i,j). For brevity, sometimes the point itself will be referenced as the (i,j) point. As will be explained next, the controller 350 of this embodiment analyzes the image intensity I at the (i,j) points according to the distance h=h(i,j) of the (i,j) points from a trial axis that forms an angle α with the x axis. Straightforward geometrical considerations show that

$$h ( i , j , α ) = {x}_{i} sin ( α ) - {y}_{j} cos ( α ) \qquad ( 1 )$$

The discrete z positions of the movable lens 332 where the images were taken are indexed by k. For specificity, the method will be described when three images are taken, typically at equidistant z positions, so k takes the values 1, 2, or 3. In this reference frame, the image intensity is a three-variable function: I=I(i,j,k), meaning that the image was captured at the movable lens position k, and the image intensity I was measured at the point (i,j) in this k-th image.

FIG. 16B recalls that when the optical system has an astigmatism (because of the patient's eye, or the setting of the rotatable Stokes lens 334, or a combination of these two), then the image spot is an elongated ellipse when the system is not at best focus. For simplicity, in the following discussion the astigmatism of rotatable Stokes lenses 334 is set to zero. As discussed e.g. in relation to FIGS. 13A-C, as the movable lens 332 is moved along the z axis, i.e. its k index is increased in the present notation, the long axis of the image spot can be aligned with the axis of the astigmatism before the optimal k position k<kₘᵢₙ, and flips to be perpendicular to it for k>kₘᵢₙ. Alternatively, the long axis may evolve from perpendicular to aligned, depending on the sign of the direction of motion of the movable lens 332 relative to the patient's eye. The controller 350 determines the axis direction and magnitude of the astigmatism from the analysis of these elongated spots, as described next.

In FIG. 16B, the elliptical spot qualitatively indicates the loci of the image pixels (i,j) in the k-th image where the light intensity I(i,j,k) exceeds some threshold. The axis representing the light intensity I(i,j,k) is meant to point out of the x-y plane of FIG. 16B. In general, the axis of the patient astigmatism has an angle β with the x axis. Broadly speaking, the controller 350 analyzes these images by rotating the angle α of the trial axis and determining how well the trial axis is aligned with the patient astigmatism axis, i.e. how close is the angle α to the angle β. If the alignment of these two axes is good, then the average distance, more precisely, the root-mean-square (RMS) distance, of the high intensity image points from the trial axis is low. If the alignment is poor, then the RMS distance of the high intensity image points from the trial axis is high. One way to calculate this RMS distance is to weigh the distance h(i,j) of each image point from the trial axis with the corresponding image intensity I(i,j,k). To manage the sign of the distance h(i,j), its magnitude or its square can be used. Finally, the distance squared, weighted by the light intensity, can be summed up for every image point to define an integrated misalignment measure S(k,α) as:

$$S ( k , α ) = {[ \frac{{\sum }_{i , j} I ( i , j , k ) \cdot {h}^{2} ( i , j , α )}{{\sum }_{i , j} I ( i , j , k )} ]}^{1 / 2} \qquad ( 2 )$$

The search for the optimal alignment of the trial axis and the patient axis is pursued in the two dimensional (k,α) space. It can be pursued at a fixed k as a function of a, or at a fixed a as a function k, or by a two dimensional search algorithm. FIG. 16C shows the search at a fixed a as a function of k. Operationally, this is performed by sweeping the movable lens 332 along a set of k stops and analyzing the images with a fixed trial axis angle α. In this k sweep S(k,α) exhibits a minimum at a kₘᵢₙ, as shown. In practice, S(k,α) can be measured only at a few k points, and then the minimum of the S(k,α) function with respect to can be found by interpolation. The interpolation can be quadratic, or spline, or any of the many known interpolation techniques. Typically, kₘᵢₙ will not be any of the measured k points, but somewhere in between. In the shown example, kₘᵢₙ is about 2.5.

FIG. 16D shows the plot of kₘᵢₙ as a function of a. Visibly, kₘᵢₙ(a) follows an approximately sinusoidal curve, with a period of 180 degrees. It can be thought of as following as a sin(2α) function. The trial axis angle where the kₘᵢₙ(a) function has its minimum is denoted by αₘᵢₙ, and where the kₘᵢₙ(a) function has its maximum is denoted by αₘₐₓ. Visibly, amin and αₘₐₓ are separated by 90 degrees. The trough and peak of this sine wave represent the focal positions k for the high power meridian and the low power meridian of the refraction. If there is no astigmatism, then kₘᵢₙ(α) is a horizontal line. The full S(k,α) function over the two dimensional (k,α) space is a sinuous valley with its valley bottom given by kₘᵢₙ(α), while the walls of the valley rise laterally from the valley bottom.

FIG. 16E shows two cross sections of this two dimensional S(k,α) surface, taken at the fixed angles, or, equivalently, along the two meridians, of αₘᵢₙ and αₘₐₓ, as a function of k. Both cross sections, S(k,αₘᵢₙ) and S(k,αₘₐₓ), exhibit minima at their corresponding movable lens positions kₘᵢₙ(αₘᵢₙ) and kₘᵢₙ(αₘₐₓ), denoted by kₕᵢ and k_{low}, respectively, that represent the estimated positions k where the high optical power Pₕᵢ and the low optical power P_{low} of the combined optical system occur, consisting of the autorefractor 300 and the patient prescription, i.e. the patient power differential from its optimal value. The average, or midpoint, of these powers corresponds to the net spherical equivalent power of the combined optical system of the patient prescription and the autorefractor 300 that is attempting to compensate this prescription:

$$P ( residual sphere ) = {P}_{R} ( sphere ) = ( {P}_{hi} + {P}_{low} ) / 2 \qquad ( 3 )$$

The previous discussion related to FIGS. 11-15C was aimed at compensating the patient prescription with the autorefractor 300 by minimizing the image spot size, or, more generally, by optimizing the compensation indicator, and thereby reducing P_{R}(sphere)=P(patient prescription)−P(autorefractor) toward zero.

In the here-described astigmatism-related embodiment, (Pₕᵢ−P_{low}) is the measured amount of the residual, uncompensated cylinder:

$$P ( residual cylinder ) = {P}_{R} ( cylinder ) = {P}_{hi} - {P}_{low} \qquad ( 4 )$$

This P_{R}(cylinder) can be used as an additional compensation indicator, since it describes the difference between the cylinder power of the patient eye and that of the autorefractor 300 that is attempting to compensate this cylinder. This description was started with the cylinder of the autorefractor 300 set to zero. In this setup, P_{R}(cylinder)=P(patient cylinder), the cylinder power of the eye of patient. (Here the entire patient cylinder is used instead of a putative “prescription”, as the optimal patient cylinder value is zero.) Further, the angle αₘᵢₙ is simply the angle of the axis of the cylinder of the eye of the patient αₘᵢₙ=β(patient cylinder)—with the notation of FIG. 16B, since the cylinder of the autorefractor 300 is set to zero, and thus has no axis either. Some treatises use a different convention in which αₘₐₓ may denote this axis.

In the general case when the cylinder of the autorefractor 300 is non-zero, P_{R}(cylinder)=P(patient cylinder)−P(autorefractor cylinder); and α_{R,min} is the axis of the astigmatism differential between the patient astigmatism and the autorefractor astigmatism: α_{R,min}=β(patient cylinder)−β(autorefractor cylinder).

To sum, for non-astigmatic cases it was sufficient to use the spot size was as the sole compensation indicator. Minimizing the spot size zeroed out P_{R}(sphere) and this was sufficient to determine the patient sphere prescription. In the here-described astigmatic cases, additional compensation indicators are needed such as the residual quantities P_{R}(cylinder) and α_{R,min}, used together with the previously described P_{R}(sphere)-minimizing spot size.

In operation, a first run is performed as described above, and the controller 350 determines the compensation indicators P_{R}(sphere), P_{R}(cylinder) and α_{R,min}. From these, the controller 350 determines the P(patient sphere prescription), P(patient cylinder), and β(patient cylinder). Based on these, the controller 350 adjusts the rotatable Stokes lenses 334 and the position where the movable lens 332 is stopped as the central step of 3 or 5 steps, all this to compensate the detected patient power and astigmatism. Then the controller 350 performs the above steps again. If the controller 350 determined the P(patient sphere prescription), P(patient cylinder), and β(patient cylinder) values well from the first measurement, then in the second run, the P_{R}(sphere) will be approximately zero, and thus the image spot will be spherical with the minimal spot size in the central image (e.g. in second image if the movable lens 332 was stopped at three stops, or in the third image if the movable lens 332 was stopped at five stops), and the residual measured astigmatism will vanish because the autorefractor 300 was adjusted to exactly compensate for the patient's cylinder. This second run therefore can be used to validate the P(patient sphere prescription), P(patient cylinder), and β(patient cylinder) values, determined from the first run. If the second run discovers some small residual differences, then the controller 350 can adjust the autorefractor 300 for a second time to optimally compensate the P(patient sphere prescription), P(patient cylinder), and β(patient cylinder).

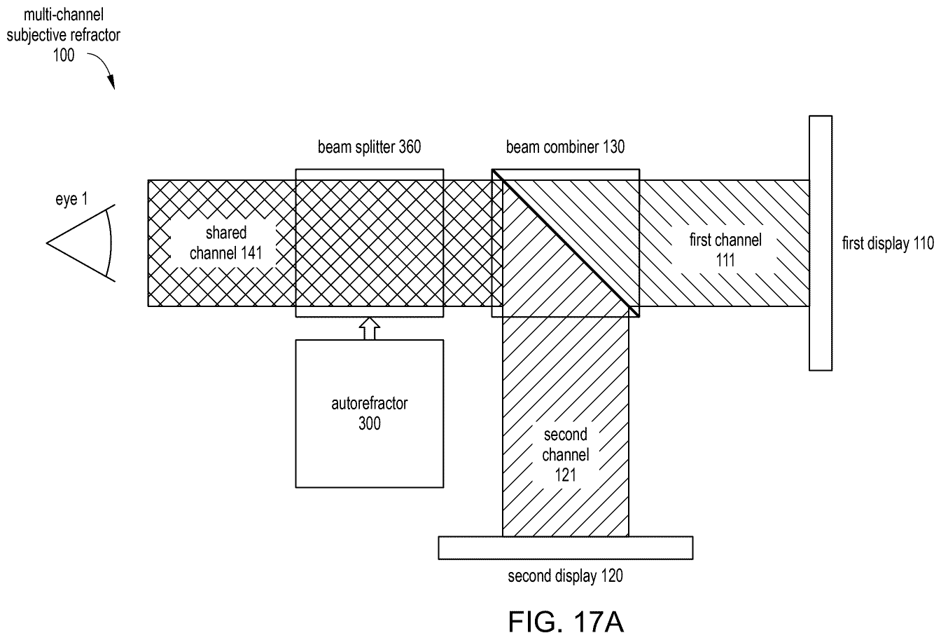

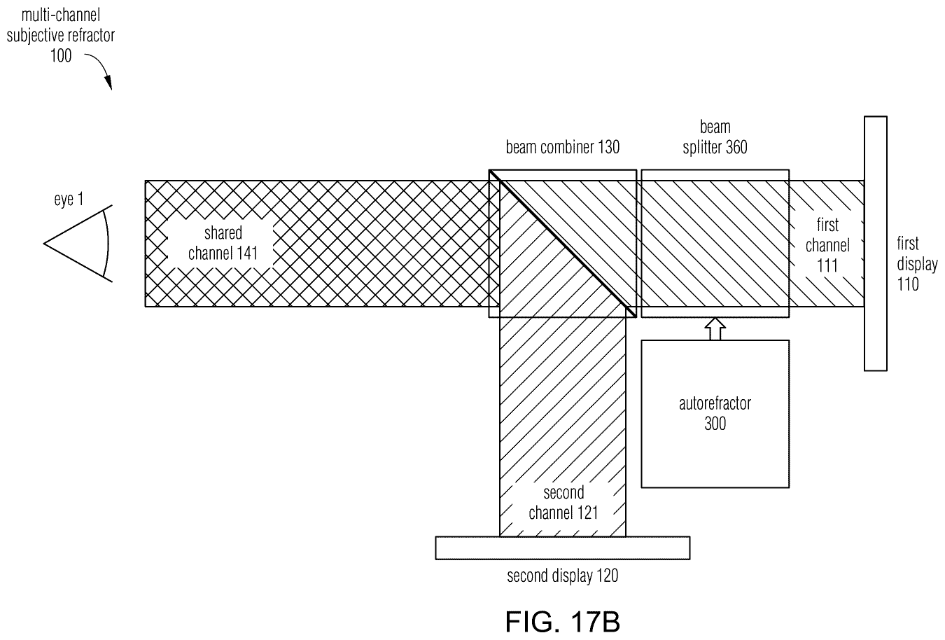

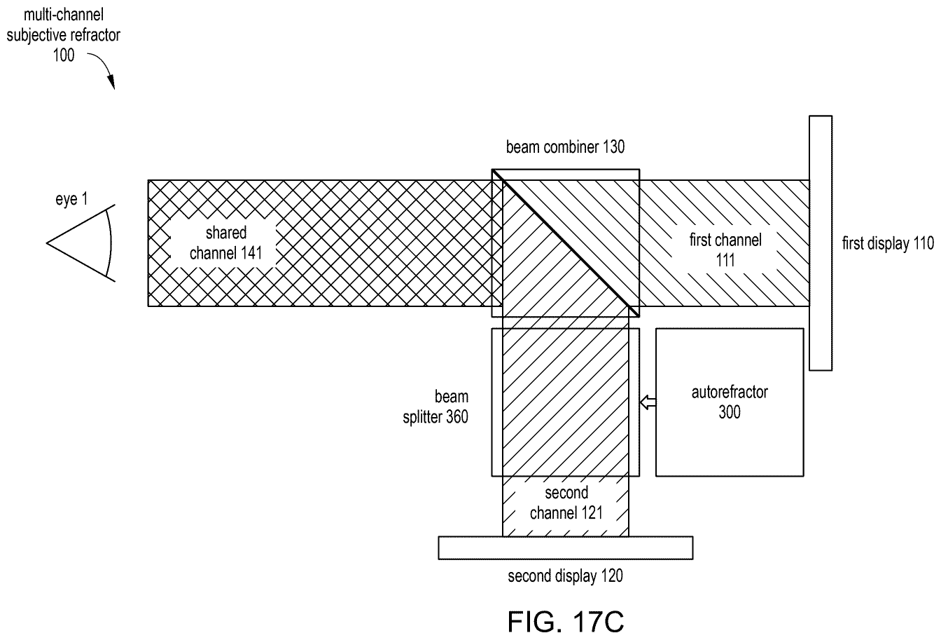

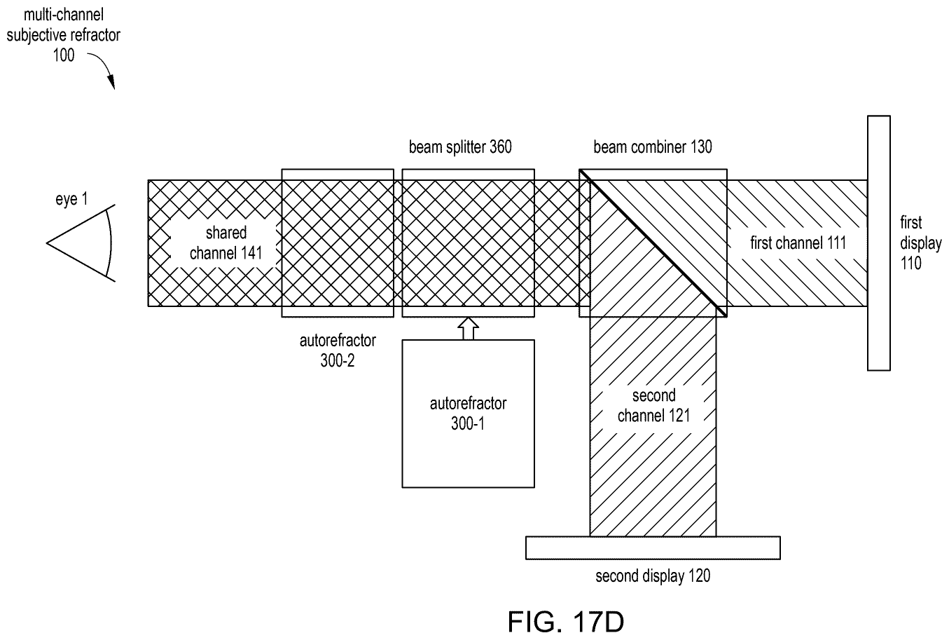

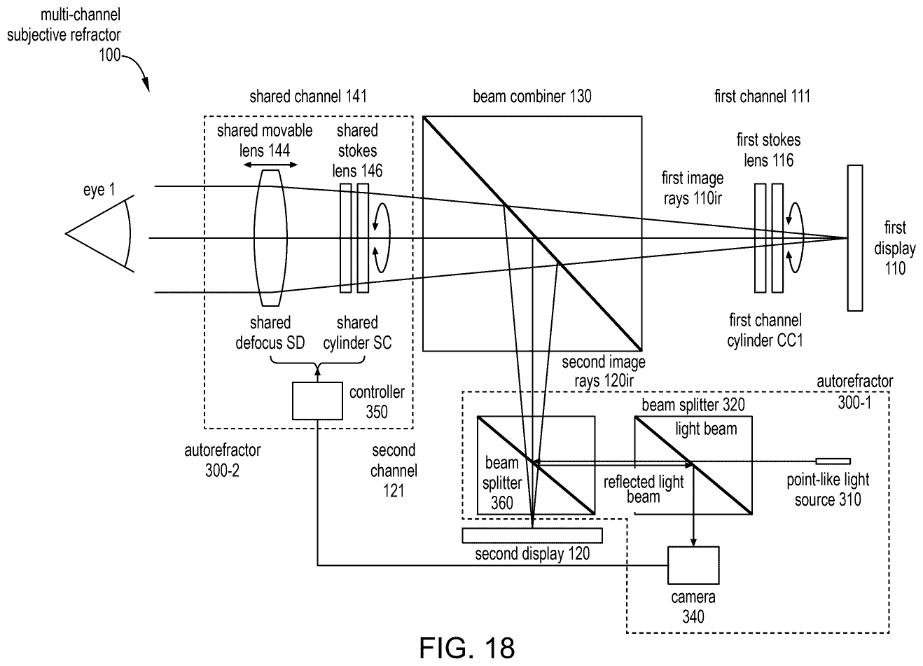

FIGS. 17A-D and FIG. 18 show how embodiments of the objective autorefractor 300 can be combined with embodiments of the multi-channel subjective refractor 100. Any of the embodiments of FIGS. 10-11 of the autorefractor 300 can be combined with any of the embodiments of FIGS. 1-7 of the multi-channel subjective refractor 100.

Embodiments of the multi-channel subjective refractor 100 have been described before in relation to FIGS. 1-9. These embodiments can include a first display 110 to generate a first image; a second display 120 to generate a second image; a first channel 111 to refract the first image with a first channel refraction; a second channel 121 to refract the second image with a second channel refraction; a beam combiner 130 to receive and to combine the first image and the second image; and a shared channel 141, to receive the first image and the second image from the beam combiner 130; to refract, in combination with the first channel 111, the first image with a first refraction; to refract, in combination with the second channel 121, the second image with a second refraction; and to present the first image with the first refraction and the second image with the second refraction to the eye 1 simultaneously.

The autorefractor 300 can be combined with the multi-channel subjective refractor 100 with a beam splitter 360. In embodiments where the operating wavelength of the autorefractor 300 is in the infrared, the autorefractor 300 can be combined with the multi-channel subjective refractor 100 via a wavelength-selective beam splitter 360. In some of these designs, the point-like light source 310 of the autorefractor 300 can operate with an infrared light; and the first display 110 and the second display 120 of the multi-channel subjective refractor 100 can operate with a visible light. As above, the infrared light of the point-like light source 310 can have a wavelength longer than 650 nm, 700 nm, or 800 nm, and less than an upper limit in the range of 100 microns-1,000 microns.

In embodiments, the wavelength-selective beam splitter 360 can be positioned in the first channel 111, in the second channel 121, or in the shared channel 141. Sometimes such wavelength-selective beam splitters 360 are called hot beam-splitters 360. FIG. 17A shows an embodiment where the beam splitter 360 is positioned in the shared channel 141. FIG. 17B shows an embodiment where the beam splitter 360 is positioned in the first channel 111. FIG. 17C shows an embodiment where the beam splitter 360 is positioned in the second channel 121.

The here-described autorefractor 300 can be advantageously combined with the multi-channel subjective refractor 100. For example, the multi-channel subjective refractor 100 and the aberration compensator optic 330 can share at least one optical element. FIG. 17D shows that in such “shared-optics” embodiments, the autorefractor 300 can be thought of as including two parts: the autorefractor part 300-1 may be separate from the multi-channel subjective refractor 100, while the shared optical elements may form the autorefractor part 300-2, where the optical elements have a dual role: they perform a function for the operation of the autorefractor 300 and a different function for the multi-channel subjective refractor 100. In every hereto described embodiments, the “combination” of the two refractors may mean that they are not completely separate, but, rather, share some optical elements.

FIG. 18 shows such a “shared-optical element” embodiment in more detail. The multi-channel subjective refractor 100 is a variant of embodiments illustrated in FIGS. 3C-F, whose description will not be repeated here. The autorefractor part 300-1 is coupled into the second channel 121 via the beam splitter 360. This autorefractor part 300-1 includes the point-like light source 310, the beam splitter 320, and the camera 340. Notably, the aberration compensator optic 330 is completely shared: it is entirely within the autorefractor part 300-2, and thus also performs the function of the shared channel 141. As shown, the controller 350, also included in the autorefractor part 300-2, can be configured to adjust the shared movable lens 144 that also functions as the movable lens 332, and to adjust the shared Stokes lens 146 that also functions as the rotatable Stokes lens 334.

The autorefractor 300 can be combined with the multi-channel subjective refractor 100 in a large number of ways. All of these combinations will be understood to be within the scope without express description. For example, the movable lens 332 can be separate from the multi-channel subjective refractor 100, but the rotatable Stokes lens 334 can be in one of the channels, wherein the shared channel 141 does not contain further optical elements. Notably, in these combinations the multi-channel subjective refractor 100 contains no optical element distal to a distal end of the autorefractor 300. In such combinations, the determined optimal compensating aberration of the aberration compensator optic 330 can be directly used for a prescription for the patient. In combinations which have additional optical elements distal to the aberration compensator optic 330, the optimal aberration needs to be modified to arrive at the patient prescription.

While this document contains many specifics, details and numerical ranges, these should not be construed as limitations of the scope of the invention and of the claims, but, rather, as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to another subcombination or a variation of a subcombinations.

1. An autorefractor, comprising:
a point-like light source, to emit a light beam;
a beam splitter, to direct the light beam;
an aberration compensator optic,
to receive the light beam from the beam splitter,
to propagate the light beam with a compensating aberration to an eye of a patient, and
to propagate a reflected light beam, reflected by the eye, to the beam splitter; wherein the beam splitter is configured to direct the reflected light beam, received from the aberration compensator optic;
a camera,
to receive the reflected light beam from the beam splitter, and
to capture an image formed from the reflected light beam; and
a controller,
to determine a compensation indicator of the reflected light beam from the captured image, and
to adjust the aberration compensator optic to improve the compensation indicator.

2. The autorefractor of claim 1, wherein:
the point-like light source is a tip of an optical fiber, a laser, a luminous disk, an LED, or an LED with a pinhole in front of it.

3. The autorefractor of claim 1, wherein:
the diameter of the point-like light source is less than 2 mm, 1 mm, or 0.5 mm.

4. The autorefractor of claim 1, wherein:
the point-like light source emits the light beam as an infrared light with a wavelength exceeding 650 nm.

5. The autorefractor of claim 1, wherein:
the beam splitter is configured
to redirect the light beam from the point-like light source toward the aberration compensator optic; and
to transmit the reflected light beam from the eye to the camera.

6. The autorefractor of claim 5, wherein:
the beam splitter is a polarizing beam splitter; and
the point-like light source is configured to emit the light with an s-wave polarization, defined relative to a diagonal surface of the beam splitter.

7. The autorefractor of claim 1, wherein:
the beam splitter is configured
to transmit the light beam from the point-like light source toward the aberration compensator optic; and
to redirect the reflected light beam from the eye to the camera.

8. The autorefractor of claim 7, wherein:
the beam splitter is a polarizing beam splitter; and
the point-like light source is configured to emit the light with a p-wave polarization, defined relative to a diagonal surface of the beam splitter.

9. The autorefractor of claim 1, wherein:
the aberration compensator optic is configured to impart a compensating defocus by including at least one of
a movable lens, slidably movable by a translation stage;
a movable lens group, wherein at least one lens of the lens group is slidably movable by a translation stage;
a variable power lens that is variable by mechanical, fluidic, or electro-optical means; and
a variable power reflective optics.

10. The autorefractor of claim 1, wherein:
the aberration compensator optic is configured to impart a compensating astigmatism by including a rotatable Stokes lens pair.

11. The autorefractor of claim 1, wherein:
optical path lengths from a proximal surface of the aberration compensator optic to the point-like light source and to the camera are the same.

12. The autorefractor of claim 1, wherein:
the compensation indicator is a spot size of the reflected light beam in the image captured by the camera; and
the controller is configured to adjust the aberration compensator optic to minimize the spot size.

13. The autorefractor of claim 12, wherein:
the spot size is minimal when an aberration of the aberration compensator optic compensates an aberration the eye of the patient, within a tolerance.

14. The autorefractor of claim 13, wherein:
the aberration of the aberration compensator optic is a prescription of the patient.

15. The autorefractor of claim 13, wherein:
the aberration of the aberration compensator optic includes a power, a cylinder, and an axis of the cylinder, and each of these optimally compensate a power, cylinder, and axis of the cylinder of the eye of the patient when the spot size is minimal, within a tolerance.

16. The autorefractor of claim 13, wherein:
a prescription for the patient is determined by correcting an optimally compensating aberration of the aberration corrector optic for a dispersion of a refractive index of the eye of the patient by at least one of a defocus of the camera, a defocus of the point-like light source, a combination of these, or a software of the controller.

17. The autorefractor of claim 1, wherein:
the compensation indicator is related to a spot of the reflected light beam in the image captured by the camera; and
the controller is configured to adjust the aberration compensator optic to minimize the spot size, to minimize an ellipticity of the spot, to sharpen a contour of the spot, or to maximize a brightness of the spot.

18. The autorefractor of claim 1, wherein:
the aberration compensator optic is configured to compensate a higher order aberration of the eye of the patient, within a tolerance.

19. The autorefractor of claim 1, wherein:
the controller is configured to improve the compensation indicator in an iterative manner, in order to eventually optimize the compensation indicator.

20. The autorefractor of claim 1, comprising:
a variable power lens, with an optical power that can be oscillated by the controller so that a size of a spot, imaged by the camera, oscillates; and
a lock-in amplifier, part of the controller, to filter out the oscillating spot size from the camera image; wherein
the spot size is used as the compensation indicator.

21. The autorefractor of claim 20, wherein:
the controller is configured
to determine a direction and, optionally, a rate of change of the spot size as a function of the optical power of the variable power lens, based on the oscillating spot size filtered out by the lock-in amplifier; and
to adjust the aberration compensator optic based on the determined direction and optional rate of change of the spot size.

22. The autorefractor of claim 1, wherein:
the aberration compensator optic includes
a movable lens; and
a rotatable Stokes lens pair; and
the controller is configured
to move the movable lens through a set of stops so as to capture a sequence of images at these stops;
to determine an RMS distance of image points of the image sequence from a trial axis, weighted with the image intensity;
to determine compensation indicators from analyzing the RMS distance as a function of a trial axis angle and a movable lens stop index;
to move the movable lens and to rotate the rotatable Stokes lens to optimize the compensation indicators; and
to determine a patient sphere prescription, a patient cylinder and a patient astigmatism axis from these determined optimal compensation indicators.

23. The autorefractor of claim 1, wherein:
the autorefractor is combined with a multi-channel subjective refractor, comprising
a first display to generate a first image;
a second display to generate a second image;
a first channel to refract the first image with a first channel refraction;
a second channel to refract the second image with a second channel refraction;
a beam combiner to receive and to combine the first image and the second image; and
a shared channel,
to receive the first image and the second image from the beam combiner;
to refract, in combination with the first channel, the first image with a first refraction;
to refract, in combination with the second channel, the second image with a second refraction; and
to present the first image with the first refraction and the second image with the second refraction to an eye simultaneously.

24. The autorefractor of claim 23, wherein:
the autorefractor is combined with the multi-channel subjective refractor via a wavelength-selective beam splitter; wherein
the wavelength-selective beam splitter is positioned in the first channel, in the second channel, or in the shared channel.

25. The autorefractor of claim 24, wherein:
the point-like light source of the autorefractor operates with an infrared light; and
the first display and the second display of the multi-channel subjective refractor operates with a visible light.

26. The autorefractor of claim 25, wherein:
the infrared light of the point-like light source of the autorefractor has a wavelength longer than 650 nm, 700 nm, or 800 nm.

27. The autorefractor of claim 23, wherein:
the multi-channel subjective refractor and the aberration compensator optic share at least one optical element.

28. The autorefractor of claim 23, wherein:
the multi-channel subjective refractor contains no optical element distal to a distal end of the autorefractor.
